(19) United States
(12) Patent Application Publication
(10) Pub. No.: US 2024/0050475 A1
(43) Pub. Date: Feb. 15, 2024

(54) COMPOSITION COMPRISING AN ANTIBODY WHICH BINDS TO HUMAN PRDX4 PRESENT ON THE CELL SURFACE OF A TARGET CELL

(71) Applicants: Helmholtz Zentrum München Deutsches Forschungszentrum für Gesundheit und Umwelt (GmbH), Neuherberg (DE); Eximmium Biotechnologies GmbH, Munich (DE)

(72) Inventor: Reinhard Zeidler, Olching (DE)

(21) Appl. No.: 18/266,446

(22) PCT Filed: Dec. 10, 2021
(86) PCT No.: PCT/EP2021/085162

§ 371 Date: Jun. 9, 2023

(30) Foreign Application Priority Data
Dec. 10, 2020 (LU) 102276
Feb. 8, 2021 (EP) 21155773.1

(51) Int. Cl. A61K 35/17; A61K 39/00; C07K 16/40; C07K 16/30; A61P 35/00

(52) U.S. Cl. CPC A61K 35/17; A61K 39/4631; C07K 16/40; C07K 16/3069; A61P 35/00; A61K 2239/13

(57) ABSTRACT

The present invention provides a pharmaceutical composition comprising an antibody which binds to human PRDX4 present on the cell surface of a target cell and optionally a pharmaceutically acceptable carrier, diluent or excipient. The present invention further provides said pharmaceutical composition for use in a method for the treatment of cancer in a human subject. Further is provided a method for determining whether a human subject may suffer from cancer, comprising determining in a sample obtained from said human subject whether PRDX4 is present on the cell surface of cells comprised by said sample. Additionally, an antibody which binds to human PRDX4 present on the cell surface of a target cell is provided.

### TECHNICAL FIELD OF THE INVENTION

The present invention provides a pharmaceutical composition comprising an antibody which binds to human PRDX4 present on the cell surface of a cancer cell and optionally a pharmaceutically acceptable carrier, diluent or excipient. The present invention further provides said pharmaceutical composition for use in a method for the treatment of cancer in a human subject. Further is provided a method for determining whether a human subject may suffer from cancer, comprising determining in a sample obtained from said human subject whether PRDX4 is present on the cell surface of cancer cells comprised by said sample. Additionally, an antibody which binds to human PRDX4 present on the cell surface of a target cell is provided.

### BACKGROUND ART

It has long been known that tumor cells carry proteins on their surfaces that are only found inside and/or are secreted in non-tumor cells. In normal cells, these proteins are often located in the membranes of the Golgi apparatus or of the endoplasmic reticulum (ER). It is not known why and for what purpose tumor cells carry them on their surface (Weidle et al., 2011). Little is known about the function of such proteins on the surface of tumor cells. On the other hand, these proteins can be of interest as target structures for certain applications, as they may represent tumor-associated or -specific antigens that can be controlled with various methods. These include, e.g., antibodies such as can be used for the detection or treatment of (tumor) diseases.

Peroxiredoxin IV (PRDX4; UniProt Q13162) is a member of the PRDX family, which consists of six members. PRDX4 is found in relatively high concentrations in most, if not all, cells. Its function is to convert H₂O₂ to water and thereby reduce oxidative stress in cells. PRDX4 is localized within cells mainly in the lumen of the ER. A secreted form of PRDX4 has also been described (Okado-Matsumoto et al., 2000, #88382).

PRDX4 is overexpressed in many tumor types and is often a marker of a poor clinical prognosis (Hwang et al., 2015; Jeong et al., 2020; Mizutani et al., 2019; Schulte et al., 2011; Schulte, 2011; Wang et al., 2019). However, it has so far not been described as being on the surface of tumor cells. Indeed, Hwang et al., 2015 uses polyclonal antibodies against PRDX4 in immunohistochemical stainings which require that the cells of the tissue to be examined are permeabilized, since PRDX4 is a known intracellular protein. Accordingly, Hwang et al., 2015 applies to formalin-fixed, paraffin-embedeed tissue sections a heat epitope retrieval by incubating the tissue sections with ethylene diamine in order to make intracellular epitopes available for being bound by antibodies. Yet, Hwang et al., 2015 could not detect PRDX4 on the cell surface of cancer cells. As a result, Hwang et al., 2015 fails to teach antibodies which bind to human PRDX4 present on the cell surface of a cancer cell for use a medicament, e.g. for treating cancer.

EP-A1 2546269 provides antibodies against PRDX4 for use as a diagnostic marker or for therapeutic treatment of the focus of a necrosis. Antibodies were prepared against the amino acid sequence WETEERPRT (SEQ ID NO: 82) of PRDX4 which was found to be present in cell extracts of necrotic HeLa cells. The aformentioned amino acid sequence corresponds to amino acid positions 38-46 of SEQ ID NO: 81. It was observed in EP-A1 2546268 that such antibodies do not recognize live HeLa cells (see Example 8.2), i.e., the antibodies did not bind to intact HeLa cells and it was concuded that this is so, because PRDX4 is an intracellular protein. Such antibodies however stained in immunohistochemical assays necrotic areas within tissue sections of tumors caused by HeLa cells in a mouse model. It is known that in necrosis, intracellular proteins are released and are accessible for antibodies. EP-A1 2546268 could not detect PRDX4 on the cell surface of cancer cells. Accodingly, EP-A1 2546268 fails to teach antibodies which bind to human PRDX4 present on the cell surface of a cancer cell for use a medicament, e.g. for treating cancer.

Monoclonal antibodies (AK) against human PRDX4 were developed from immunizations that were carried out in cooperation with the core facility ‘Monoclonal Antibodies’ at the Helmholtz Center Munich (HMGU). The inventors have proven the specificity of these AKs in various experiments of the present invention. These antibodies were found to bind to the surface of various human tumor cell lines.

Thus, the present invention is the first time that PRDX4 has been described to be carried on the surface of tumor cells. The mentioned antibodies specifically recognize the PRDX4 on the surface of tumor cells and provides novel tumor-specific target-molecules for cancer therapy.

### SUMMARY OF THE INVENTION

The present invention provides a pharmaceutical composition comprising an antibody, preferably a monoclonal antibody, which binds to human PRDX4 present on the cell surface of a cancer cell, more preferably said antibody recognizes a region from amino acids 81 to 271 in human PRDX4 shown in SEQ ID NO: 81 and optionally a pharmaceutically acceptable carrier, diluent or excipient.

In one embodiment of the pharmaceutical composition of the present invention, said antibody has cytotoxic activity.

In one further embodiment of the pharmaceutical composition of the present invention, said antibody has ADCC or CDC.

In one embodiment of the pharmaceutical composition of the present invention, said antibody is conjugated with a cytotoxic substance.

In one further embodiment of the pharmaceutical composition of the present invention, said antibody is part of a chimeric antigen receptor (CAR). For said embodiment, it is preferred that said CAR is comprised by a T cell, NK cell, NK-T cell or macrophage.

In one embodiment of the pharmaceutical composition of the present invention, said antibody is less than 20% cross-reactive with PRDX4-related proteins.

In one further embodiment of the pharmaceutical composition of the present invention, said antibody recognizes a region from amino acids 1 to 79, preferably from amino acids 37 to 79, more preferably from amino acids 81 to 271, in human PRDX4 shown in SEQ ID NO: 81.

Additionally, in one embodiment of the pharmaceutical composition of the present invention, said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16; an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(i) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

In one embodiment of the pharmaceutical composition of the present invention, said antibody is
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23, and
   a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
   a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
   a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
   a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
   a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
   a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
   a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62,
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
   a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68,
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
   a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74,
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
   a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80, or
(k) an antibody which binds to the same epitope as that in the human PRDX4 protein to which any one of the antibodies (a) to (j) bind.

In a further aspect, the present invention provides the pharmaceutical composition according to the present invention for use in a method for the treatment of cancer in a human subject. It is preferred for said embodiment, that said cancer is characterized by cells, wherein human PRDX4 is present on their cell surface. It is also preferred for said embodiment that said cancer is breast cancer, prostate cancer, lung cancer, colorectal cancer, ovarian cancer, oral cavity squamous carcinoma, hematologic malignancy tumor, or glioma.

In a further aspect, the present invention provides a method for determining whether a human subject may suffer from cancer, comprising determining in a sample obtained from said human subject whether PRDX4 is present on the cell surface of cancer cells comprised by said sample.

In a further aspect, the present invention provides an antibody which binds to human PRDX4 present on the cell surface of a target cell, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16;
(i) an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(j) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

In one further embodiment, the present invention provides an antibody which binds to human PRDX4 present on the cell surface of a target cell comprising
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23 and
   a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
   a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
   a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
   a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
   a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
   a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
   a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62;
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
   a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68;
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
   a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74;
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
   a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80; or
(k) an antibody which binds to the same epitope as that in the human PRDX4 protein to which any one of the antibodies (a) to (j) bind.

### BRIEF DESCRIPTION OF THE DRAWINGS

FIG. 1 shows that PRDX4 is a hypoxia-induced protein on the surface of human cancer cells. FIG. 1a) shows A549 lung cancer cells and ES-2 ovarian cancer cells that were cultured in standard cell culture medium under normoxic (approx. 18% O₂) or hypoxic (1% O₂) conditions. The expression of surface PRDX4 was measured by flow cytometry using the antibody 11F2 as a primary antibody and a Alexa647-labelled goat-anti-rat IgG as a secondary antibody. FIG. 1b) shows the measured mean fluorescence intensities from FIG. 1a). Given are the mean values and standard deviations from three independent experiments. No expression was observed on primary PBMCs, hepatocytes and cardiac fibroblasts (data not shown).

FIG. 2 shows in FIG. 2a) that PRDX4 is expressed on various human cancer cell lines of different origin and on cancer cells isolated from primary ascites from patients with ovarian cancer. Row 1 shows ovarian cancer cell lines. Row 2 shows primary ascites from ovarian cancer. Row 3 shows colon, cervix and lung carcinoma, mesothelioma. Row 4 shows HEK293 cells. Row 5 shows negative cell lines derived from a T cell leukemia, a head and neck cancer and primary peripheral blood mononuclear cells (PBMCs) from a healthy donor. FIG. 2b) shows that the PRDX4-specific antibody 11F2 precipitates PRDX4 from lysates of cell lines that express surface PRDX4 or not. Cyanogen bromide beads were coupled with 11F2 and then incubated with 0.5 mg lysate in RIPA buffer at 4° C. overnight and then centrifuged at 3.000 rpm for 5 min. Precipitated beads were incubated with Laemmli buffer, boiled for 5 min, and the eluates were separated by SDS-PAGE (lanes ‘IP’). Cell lysats were used as controls (L). Gels were blotted and the blots were incubated with a commercial PRDX4 antibody (Thermo Scientific), then with a HRP-labelled goat-anti rat antibody and developed with ECL. FIG. 2c) shows that PRDX4 is biotinylated and thus present on the surface of cancer cells. Vital ES-2 tumor cells were incubated with membrane-impermeable EZ-Link Sulfo-NHS-SS-Biotin (Thermo Scientific) to exclusively biotinylate surface proteins. Then, cells were lysed and the lysate was incubated with 11F2-coupled beads as described above. Beads coupled with other antibodies were used as a control. Eluates of the precipitated beads were separated by SDS-PAGE. Immunoblots were performed with a streptavidin-HRP conjugate to detect biotinylated proteins (upper part) and with a commercial PRDX4 antibody (lower part). Blots were developed with ECL. Lysates from non-biotinylated cells (−) were used as controls. The first 2 lanes are cell lysates before IP.

FIG. 3 shows that 11F2 binds to vital ES-2 cells. ES-2 cells were grown on microscope slides, washed in PBS and incubated with the PRDX4 antibody 11F2. Then, cells were fixed with 4% paraformaldehyde and incubated with an Alexa488-coupled suitable secondary antibody. Nuclei were counterstained with DAPI.

FIG. 4 shows that the antibody 11F2 precipitates PRDX4. Cyanogen bromide beads coupled with 11F2 were incubated overnight with lysates from ES-2 cells. After precipitation of the beads and incubation in Laemmli buffer, eluates were separated by SDS-PAGE and blotted. An immunoblot with a commercial PRDX4 antibody (Thermo Scientific) and a suitable secondary antibody revealed specific precipitation of PRDX4 by 11F2. Beads coupled with a FLAG-specific antibody were used as controls. L =cell lysate before immunoprecipitation.

FIG. 5 shows that PRDX4 is membrane-associated. ES-2 cells were lysed and membrane fractions M F1 and M F2 were obtained by centrifugation at 7,000×g and 16,500×g, respectively. The cytosolic fraction, MF3, was obtained by centrifugation at 100,000×g.

FIG. 6 shows that surface-PRDX4-positive SKOV3-cells activate PRDX4-specific CAR-T-cells. Primary T-cells from two donors (FI and JE) were transduced with a retroviral vector encoding a CAR construct with a single-chain construct derived from antibody 11F2. Expression of the CAR was tested by flow cytometry with a goat-anti rat IgG antibody (not shown). SKOV3 cells were incubated with transduced T cells (columns 4-6 of the blots) of non-transduced control T cells (column 1-3 of the blots) at the ratios indicated on the X-axis for 24 h. Then, interferon-gamma in the supernatants, as a marker for T-cell activation, was quantified with a commercial ELISA assay. As an additional proof of specificity, some of the co-cultures (columns 3 and 6) were pre-incubated with 11F2 antibody in order to block recognition of target cells by the T cells. This pre-incubation resulted in a reduced interferon concentration in the supernatants.

FIG. 7 shows that 11F2 is PRDX4-specific. 11F2 coupled to cyanogen bromide beads were incubated with 10 ng of recombinant PRDX4 or, as a control, with the same amount of recombinant CCDC47 protein. After precipitation, beads were incubated in Laemmli buffer and supernatants were separated by SDS-PAGE. An immunoblot with a commercial PRDX4 antibody (Thermo Scientific) and a suitable secondary antibody revealed the specificity of 11F2. The recombinant PRDX4 protein is a dimer and thus about 50 kD in size.

FIG. 8 shows that 11F2 is PRDX4-specific: ES-2 cells were transfected with PRDX4-specific siRNAs or a control siRNA. 3 days later, binding of 11F2 was measured by flow cytometry as described in FIG. 1. Shown is that the siRNA-transfected population 4BC_KO only reveals approx. 50% of 11F2 binding (mean=874) as compared to control-siRNA transfected ES-2 w cells (mean=1908). An isotype antibody was included as a control (mean=159).

FIG. 9 shows that PRDX4-specific antibodies are present in ascites from patients with ovarian cancer. HEK293-cells were transfected with an expression plasmid encoding a PRDX4-GFP fusion protein, approx. 55 kD in size. Similar amounts of lysates of transfectants and parental HEK293 cells were separated by SDS-PAGE and blotted. This blot was then incubated with ascites, followed by incubation with a goat-anti human IgG antibody and developed with ECL.

FIG. 10 shows the respective sequences of the antibodies 2C1 (SEQ ID NO: 1 and 2, respectively), 5H5 (SEQ ID NO: 3 and 4, respectively), 13B2 (SEQ ID NO: 9 and 10, respectively), 15F11 (SEQ ID NO: 11 and 12, respectively), 11F2 (SEQ DI NO: 7 and 8, respectively), 17A10 (SEQ ID NO: 13 and 14, respectively), including the light chain and heavy chain with the respective CDR1, CDR2 and CDR3 sequences.

FIG. 11 shows that 11F2 binds to the region comprising exons 2-7 of PRDX4. HEK293 cells were transiently transfected with a PRDX4-Exon 2-7 GFP fusion protein (lanes ‘1’) or with a PRDX4-full length GFP fusion protein (lanes ‘2’). Two days later, Cyanogen bromide beads were coupled with 11F2 and then incubated with 0.5 mg lysate in RIPA buffer at 4° C. overnight and then centrifuged at 3.000 rpm for 5 min. Precipitated beads were incubated with Laemmli buffer, boiled for 5 min, and the eluates were separated by SDS-PAGE. Gels were blotted and the blots were incubated either with a commercial PRDX4 antibody (Thermo Scientific), or with a GFP-specific antibody. Blots were then incubated with suitable HRP-labelled secondary antibodies and developed with ECL. The arrow in the PRDX4 blot points to the endogenous PRDX4. Exon 2-7 corresponds to amino acids 81-271 of human PRDX4 shown in SEQ ID NO: 81.

### DETAILED DESCRIPTION OF THE INVENTION

The present invention provides in a first aspect a pharmaceutical composition comprising an antibody, preferably a monoclonal antibody, which binds to human PRDX4 present on the cell surface of a cancer cell, more preferably said antibody recognizes a region from amino acids 81 to 271 in human PRDX4 shown in SEQ ID NO: 81and optionally a pharmaceutically acceptable carrier, diluent or excipient.

The present invention thus comprises the finding that PRDX4 is present on the cell surface of (cancer) cells. Accordingly, a new therapy concept is possible. Prior art characterizes PRDX4 as ER protein or sometimes as cytosolic or even secreted protein, but not for therapeutic purposes.

The amino acid sequence of PRDX4 is known in the art (UniProt Q13162). For example, the amino acid sequence of human PRDX4 is as set forth in SEQ ID NO: 81.

The PRDX4 protein used in the present invention may be a PRDX4 protein having the sequence described above or may be a modified protein having an amino acid sequence derived from the sequence described above by the modification of one or more amino acids. Examples of the modified protein having a sequence derived from the sequence described above by the modification of one or more amino acids can include polypeptides having 70% or more, preferably 80% or more, more preferably 90% or more, even more preferably 95% or more homology to the amino acid sequence. Alternatively, partial peptides of these PRDX4 proteins may be used.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example, less than 50 contiguous amino acids).

The PRDX4 protein used in the present invention is not limited by its origin and is preferably a human PRDX4 protein.

PRDX4 has an N-terminal signal sequence. PRDX4 traverses into the endoplasmatic reticulum (ER), accompanied by cleavage of said signal sequence, but then, is retained in the ER and is not secreted.

The term “antibody” as used herein and in the context of the present invention may comprise chimeric antibodies, humanized antibodies, monovalent antibodies, polyvalent antibodies, low-molecular antibodies, a diabody or a scFv.

Chimeric antibodies refer to antibodies comprising variable and constant regions of different origins ligated with each other. For example, mouse-human heterogeneous chimeric antibodies are antibodies comprising the heavy and light chain variable regions of a mouse antibody and the heavy and light chain constant regions of a human antibody. Mouse antibody variable region-encoding DNAs are ligated with human antibody constant region-encoding DNAs, and the ligation products can be incorporated into expression vectors to prepare chimeric antibody-expressing recombinant vectors. Cells transformed with these vectors (recombinant cells) can be cultured for the expression of the DNA insert to obtain the chimeric antibodies produced during the culture.

In general, the chimeric antibodies comprise non-human animal-derived antibody variable regions and human antibody-derived constant regions. By contrast, the humanized antibodies comprise non-human animal-derived antibody complementarity-determining regions (CDRs), human antibody-derived framework regions (FRs), and human antibody-derived constant regions. The humanized antibodies are also called reshaped human antibodies. Specifically, for example, humanized antibodies comprising non-human animal (e.g., mouse) antibody CDRs grafted in human antibodies are known in the art. The humanized antibodies are useful as active ingredients for a therapeutic agent of the present invention, owing to their reduced antigenicity in the human body.

Each antibody variable region usually comprises 3 CDRs flanked by 4 FRs. The CDR regions substantially determine the binding specificity of the antibody. The CDRs have diverse amino acid sequences. On the other hand, amino acid sequences constituting the FRs often exhibit high homology among antibodies having different binding specificities. Therefore, in general, the binding specificity of a certain antibody can allegedly be transplanted to other antibodies through CDR grafting.

The antibody present in a pharmaceutical composition of the present invention may encompass bivalent antibodies typified by IgG (IgG1, IgG2, IgG4, etc.) and also monovalent antibodies or polyvalent antibodies typified by IgM as long as these antibodies bind to the PRDX4 protein. The polyvalent antibody that may be present in the pharmaceutical composition of the present invention may encompass polyvalent antibodies having antigen-binding sites, all of which are the same as each other or some or all of which are different from each other.

The antibody present in the pharmaceutical composition of the present invention may also be a low-molecular antibody that encompasses an antibody fragment deficient in a portion of the whole antibody (e.g., whole IgG). Such partial deficiency of the antibody molecule is accepted as long as the resultant antibody fragment is capable of binding to the PRDX4. It is preferred that the antibody fragment employed in the pharmaceutical composition according to the present invention should contain one or both of heavy chain variable (VH) and light chain variable (VL) regions. It is also preferred that the antibody fragment employed in the pharmaceutical composition according to the present invention should contain CDRs. The number of CDRs contained in the antibody fragment employed in the pharmaceutical composition of the present invention is not particularly limited and is preferably at least 6 CDRs: heavy chain CDR1, CDR2, and CDR3 and light chain CDR1, CDR2, and CDR3.

The amino acid sequence of VH or VL can contain one or more substitution, deletion, addition, and/or insertion. Furthermore, the antibody or antibody fragment that may be employed in the pharmaceutical composition of the present invention may be deficient in a portion of one or both of VH and VL as long as the resultant antibody fragment is capable of binding to the human PRDX4. Moreover, its variable region may be chimerized or humanized. Specific examples of the antibody fragment can include Fab, Fab′, F(ab′)2, and Fv. Moreover, specific examples of the low-molecular antibody can include Fab, Fab′, F(ab′)2, Fv, scFv (single chain Fv), diabody, sc(Fv)2 (single chain (Fv)2), and scFv-Fc. In the present invention, the low-molecular antibody is preferably a diabody or sc(Fv)2. These antibody multimers (e.g., dimers, trimers, tetramers, and polymers) are also encompassed by the low-molecular antibody that may be employed in the pharmaceutical composition of the present invention.

The term “diabody” as used herein and in the context of the present invention may refer to a bivalent antibody fragment constructed by gene fusion. The diabody is a dimer comprising two polypeptide chains. Usually, each of the polypeptide chains constituting the dimer comprises heavy and light chain variable regions linked via a linker on the same chain. The linker in the diabody is generally too short to allow paring between heavy and light chain variable regions on the same chain. Specifically, the number of amino acid residues constituting the linker is, for example, approximately 5 residues. Therefore, heavy and light chain variable regions encoded on the same polypeptide chain cannot together form a single chain variable region fragment. Instead, they form a dimer by pairing with another single chain variable region fragment. As a result, the diabody has two antigen-binding sites.

The scFv, as used in the context of the present invention, may be obtained by linking heavy and light chain variable regions of the antibody. In the scFv, the heavy and light chain variable regions are linked via a linker, preferably, a peptide linker. The heavy and light chain variable regions in the scFv can be derived from any of the antibodies described in the present specification. The peptide linker that links the variable regions is not particularly limited. For example, an arbitrary single chain peptide of approximately 3 to 25 residues can be used as the linker.

The antibody as used in the pharmaceutical composition and the antibody according to the present invention may also encompass binding entities, such as lipocalins, an aptamer or an anticalin.

The antibody, which binds to human PRDX4, used in the present invention can have various formats. However, it needs to bind to the PRDX4 protein and is not particularly limited by its origin, type, shape, etc., but may have a cytotoxic activity. Specifically, an antibody can be used, such as a non-human animal-derived antibody (e.g., a mouse, rat, or camel antibody), a human-derived antibody, a chimeric antibody, or a humanized antibody as described above. The antibody, which binds to human PRDX4 used in the present invention may in one embodiment be a polyclonal or monoclonal antibody and is preferably a monoclonal antibody.

The antibody which binds to human PRDX4 used in the present invention can be obtained as a polyclonal or monoclonal antibody using means known in the art. The antibody used in the present invention is in particular preferably a mammal-derived monoclonal antibody. The mammal-derived monoclonal antibody encompasses, for example, those produced by hybridomas and those produced by hosts transformed with expression vectors containing an antibody gene by a genetic engineering approach.

The antibody which binds to human PRDX4 may be modified with various molecules such as polyethylene glycol (PEG). Further, the antibody which binds to human PRDX4 may also be modified with a chemotherapeutic agent, a radioactive chemical, or the like, having a cytotoxic activity.

Specific examples of the antibody used in the present invention, which recognizes PRDX4 present on the cell surface of a target cell and binds thereto, may include the antibodies given and described herein.

As described above, an antibody of the present invention or as used in the pharmaceutical composition of the present invention including the substitution, deletion, addition, and/or insertion of one or more amino acids is also incorporated in the scope of the present invention and may be prepared or occur naturally. Examples of a method for introducing a mutation in the polypeptide include site-directed mutagenesis (Hashimoto-Gotoh, T. et al., 1995) (Zoller, M J, and Smith, M., 1983) (Kramer, W. et al., 1984) (Kramer W, and Fritz H J, 1987) (Kunkel, T A, 1985) (Kunkel, 1988). This is one of the methods well known by those skilled in the art for preparing a polypeptide functionally equivalent to a certain polypeptide. Those skilled in the art can appropriately introduce a mutation in the antibody of the present invention or the antibody as used in the composition of the present invention using such a method and thereby prepare an antibody functionally equivalent to such antibody. Moreover, amino acid mutations may occur in the natural world. Such an antibody that has an amino acid sequence derived from the amino acid sequence of the antibody of the present invention or the antibody as used in the composition of the present invention comprising the mutation of one or more amino acids, is functionally equivalent or a variant to the antibody and is also encompassed by the antibody of the present invention or the antibody as used in the pharmaceutical composition of the present invention.

The number of amino acids mutated in such a variant is usually within 50 amino acids, preferably within 30 amino acids, more preferably within 10 amino acids (e.g., within 5 amino acids).

For amino acid residues to be mutated, it is preferred that this mutation should be performed conservatively between amino acids having the same side chain property. For example, the following classification based on the properties of amino acid side chains has been established: hydrophobic amino acids (A, I, L, M, F, P, W, Y, and V), hydrophilic amino acids (R, D, N, C, E, Q, G, H, K, S, and T), amino acids having an aliphatic side chain (G, A, V, L, I, and P), amino acids having a hydroxy group-containing side chain (S, T, and Y), amino acids having a sulfur atom-containing side chain (C and M), amino acids having a side chain containing carboxylic acid and amide (D, N, E, and Q), amino acids having a base-containing side chain (R, K, and H), and amino acids having an aromatic group-containing side chain (H, F, Y, and W) (all symbols within the parentheses represent single letter codes of amino acids).

A polypeptide having an amino acid sequence modified from a certain amino acid sequence by the deletion and/or addition of one or more amino acid residue(s) and/or the substitution thereof by other amino acids is already known to maintain the biological activity of the original polypeptide (Mark, D. F. et al., 1984) (Wang, A. et al., 1982). Specifically, when amino acids in an amino acid sequence constituting a certain polypeptide are substituted by amino acids classified in the same group thereas, it is generally said that the polypeptide is likely to maintain its activity. In the present invention, the substitution between amino acids within the same amino acid group described above is referred to as conservative substitution.

The term “surface” or specifically “cell surface” as used herein means the cell membrane. The cell membrane, which may also be known as plasma membrane, is the thin membrane that surrounds every living cell, delimiting the cell from the environment around it. Enclosed by this cell membrane are the cell's constituents, often large, water-soluble, highly charged molecules such as proteins, nucleic acids, carbohydrates, and substances involved in cellular metabolism. The cell membrane, therefore, has two functions: first, to be a barrier keeping the constituents of the cell in and unwanted substances out and, second, to be a gate allowing transport into the cell of essential nutrients and removal of waste products from the cell. ER-derived vesicles may also participate in the building or formation of the cell membrane.

The term “target cell”, as used within the context of the present invention, is preferably a cancer cell. The term “cancer”, as used herein, can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, testicular cancer, thyroid cancer, urinary system cancer, uterine sarcoma, vaginal cancer, vascular system, Waldenstrom's macroglobulinemia and Wilms' tumor.

The term “pharmaceutically acceptable carrier, diluent or excipient” as used in the context of the present invention, may comprise any pharmaceutically acceptable carrier, diluent or excipient for a pharmaceutical composition known by the person skilled in the art. It will be understood that such antibodies or pharmaceutical composition as described herein may be mixed with carriers or diluents, which will not interfere with the intended purpose of the present invention. For example, such a carrier as used within the present invention may be a carrier protein, such as bovine serum albumin (BSA) or keyhole limpet haemocyanin (KLH).

In one embodiment of the pharmaceutical composition of the present invention, said antibody has cytotoxic activity.

In the context of the present invention, the phrase “cytotoxic activity” refers to having a PRDX4 binding activity and may also include to have an activity equivalent to that of the antibody of the present invention. In the present invention, the equivalent activity is not necessarily required to be an identical activity and may be, for example, 50% or more, preferably 70% or more, more preferably 90% or more activity compared with the activity of any of the antibodies (a) to (k) as described herein. Examples of the upper limit of the activity can include, but is not particularly limited to, 1000% or less, 500% or less, 300% or less, 150% or less, and 100% or less.

In one further embodiment of the pharmaceutical composition of the present invention, said antibody has ADCC or CDC.

Thus, examples of the cytotoxic activity according to the present invention can include ADCC and CDC activities. In the context of the present invention, the ADCC activity means the activity of damaging target cells through the binding of Fcγ receptor-bearing cells (immunocytes, etc.) via the Fcγ receptors to the Fc domains of antibodies specifically attached to the cell surface antigens of the target cells. On the other hand, the CDC activity means a cytotoxic activity mediated by the complement system. Whether or not the antibody has an ADCC activity or has a CDC activity can be determined by a method known in the art.

Thus, the antibody employed in the pharmaceutical composition of the present invention may have activities such as an ADCC activity and as such, may be useful as a pharmaceutical drug, preferably, an anti-cancer agent, wherein the cancer is e.g. breast cancer, prostate cancer, lung cancer, colorectal cancer, ovarian cancer, oral cavity squamous carcinoma, hematologic malignancy tumor, or glioma.

In one embodiment of the pharmaceutical composition of the present invention, said antibody is conjugated with a cytotoxic substance.

In one preferred embodiment of the pharmaceutical composition, the antibody may be conjugated with a cytotoxic substance, such as a chemotherapeutic agent, a toxic peptide, or a radioactive chemical. Such a modified antibody (hereinafter, referred to as an antibody conjugate) can be obtained by chemically modifying the obtained antibody. A method for the antibody modification has already been established in the art.

Examples of the chemotherapeutic agent whose cytotoxic activity functions through the conjugation to the antibody that binds PRDX4 can include the following chemotherapeutic agents: azaribine, anastrozole, azacytidine, bleomycin, bortezomib, bryostatin-1, busulfan, camptothecin, 10-hydroxycamptothecin, carmustine, celebrex, chlorambucil, cisplatin, irinotecan, carboplatin, cladribine, cyclophosphamide, cytarabine, dacarbazine, docetaxel, dactinomycin, daunomycin glucuronide, daunorubicin, dexamethasone, diethylstilbestrol, doxorubicin, doxorubicin glucuronide, epirubicin, ethinyl estradiol, estramustine, etoposide, etoposide glucuronide, floxuridine, fludarabine, flutamide, fluorouracil, fluoxymesterone, gemcitabine, hydroxyprogesterone caproate, hydroxyurea, idarubicin, ifosfamide, leucovorin, lomustine, mechlorethamine, medroxyprogesterone acetate, megestrol acetate, melphalan, mercaptopurine, methotrexate, mitoxantrone, mithramycin, mitomycin, mitotane, phenyl butyrate, prednisone, procarbazine, paclitaxel, pentostatin, semustine, streptozocin, tamoxifen, taxanes, taxol, testosterone propionate, thalidomide, thioguanine, thiotepa, teniposide, topotecan, uracil mustard, vinblastine, vinorelbine, vincristine.

The chemotherapeutic agent is preferably a low-molecular chemotherapeutic agent. The low-molecular chemotherapeutic agent is unlikely to interfere with the antibody even after its conjugation to the antibody. The low-molecular chemotherapeutic agent usually has a molecular weight of 100 to 2000, preferably 200 to 1000. All of the chemotherapeutic agents exemplified above are low-molecular chemotherapeutic agents. These chemotherapeutic agents encompass prodrugs that are converted in vivo to active chemotherapeutic agents. The prodrug activation may be an enzymatic conversion or a non-enzymatic conversion.

In one further embodiment of the pharmaceutical composition of the present invention, said antibody is part of a chimeric antigen receptor (CAR). For said embodiment, it is preferred that said CAR is comprised by a T cell, NK cell, NK-T cell or macrophage.

Chimeric antigen receptors (CARs, also known as chimeric immunoreceptors, chimeric T cell receptors or artificial T cell receptors) are receptor proteins that have been engineered to give T cells the ability to target a specific protein.

In one embodiment of the pharmaceutical composition of the present invention, said antibody is less than 20% cross-reactive with PRDX4-related proteins. This means preferably that said antibody is less than 20% cross-reactive with other peroxiredoxins such as PRDX1, PRDX2, PRDX3, PRDX5 or PRDX6. More preferably, said antibody is less than 15% cross-reactive with PRDX4-related proteins, e.g. PRDX1, PRDX2, PRDX3, PRDX5 or PRDX6. Even more preferably, said antibody is less than 10% cross-reactive with PRDX4-related proteins, e.g. PRDX1, PRDX2, PRDX3, PRDX5 or PRDX6. Even more preferably, said antibody is less than 5% cross-reactive with PRDX4-related proteins, e.g. PRDX1, PRDX2, PRDX3, PRDX5 or PRDX6. Even more preferably, said antibody is less than 3% cross-reactive with PRDX4-related proteins, e.g. PRDX1, PRDX2, PRDX3, PRDX5 or PRDX6. Even more preferably, said antibody is less than 2% cross-reactive with PRDX4-related proteins, e.g. PRDX1, PRDX2, PRDX3, PRDX5 or PRDX6. Even more preferably, said antibody is less than 1% cross-reactive with PRDX4-related proteins, e.g. PRDX1, PRDX2, PRDX3, PRDX5 or PRDX6.

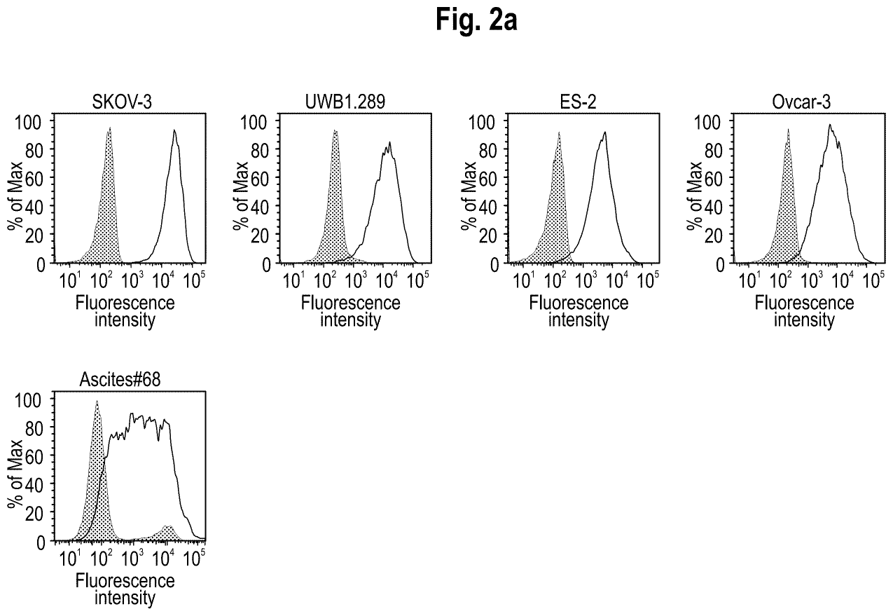

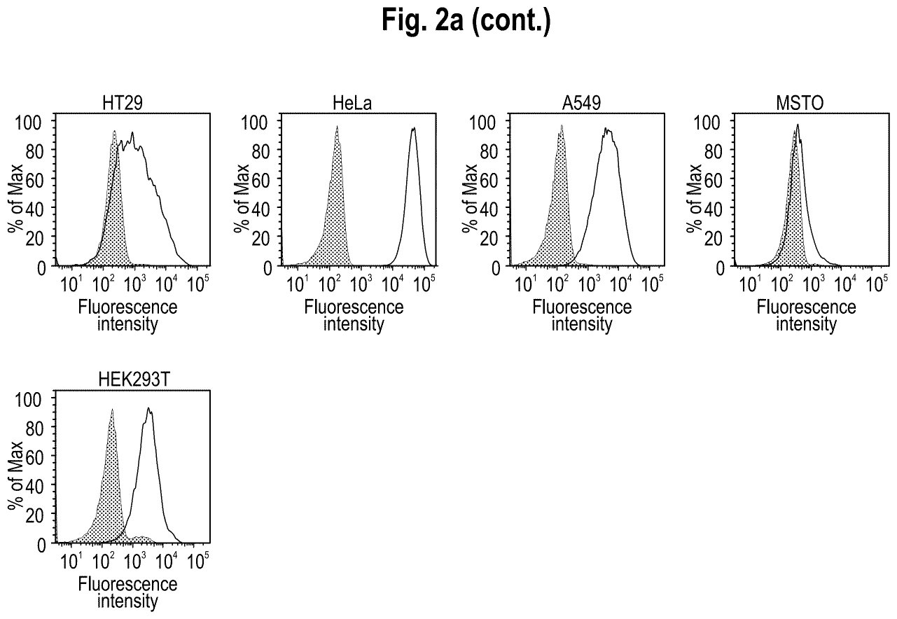

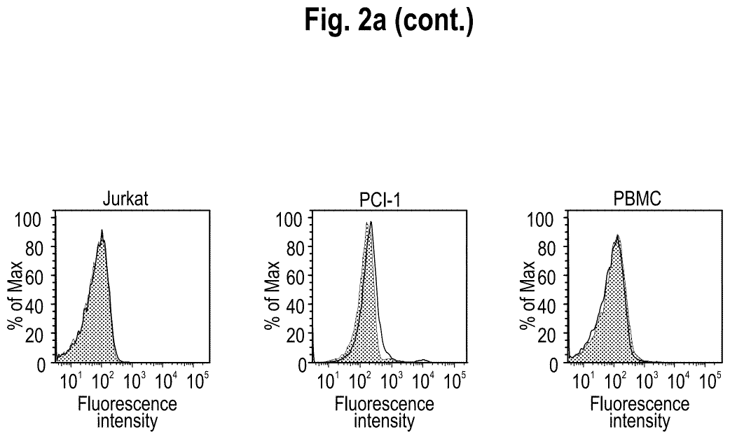

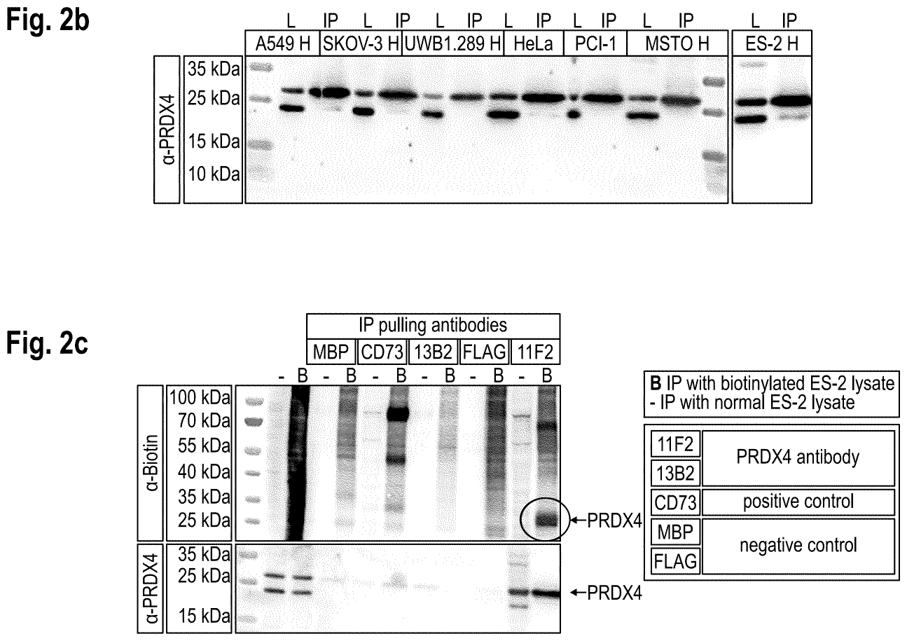

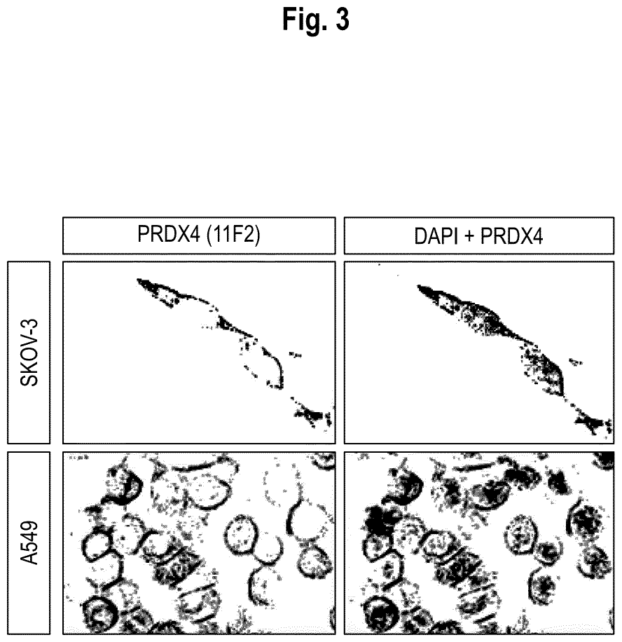

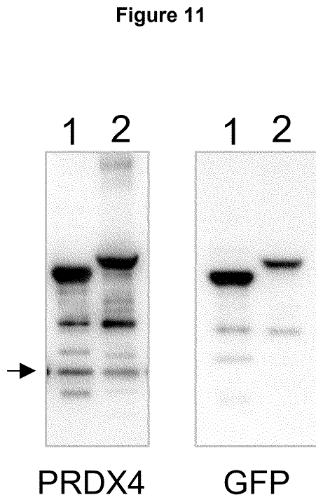

In one further embodiment of the pharmaceutical composition of the present invention, said antibody recognizes a region from amino acids 1 to 79, preferably from amino acids 37 to 79, more preferably from amino acids 81 to 271, in human PRDX4 shown in SEQ ID NO: 81 (see also FIG. 11 and its legend). As is apparent from FIG. 2A, antibodies of the present invention which recognize the more preferred region from amino acis 81 to 271 in human PRDX4 shown in SEQ ID NO: 81 bind to intact HeLa cells. In contrast, antibodies disclosed in

EP-A1 2546269 which bind to a region within amino acids 38 to 46 (shown in SEQ ID NO: 82) of human PRDX4 (shown in SEQ ID NO: 81) failed to bind to intact HeLa cells as is reported in Example 8.2 of EP-A1 2546269. Thus, it appears that the more preferred region from amino acis 81 to 271 in human PRDX4 shown in SEQ ID NO: 81, when PRDX4 is present on the cell surface of a cancer cell, provides for antibody binding thereto. Without being bound by theory, it appears that the present invention provides that region within PRDX4, i.e. the region from amino acis 81 to 271 in human PRDX4 shown in SEQ ID NO: 81, which, when PRDX4 is present on the cell surface of a cancer cell, is accessible for antibodies binding thereto.

Thus, it appears that the present invention not only provides for the first time PRDX4 as neo-cell surface protein on the cell surface of cancer cells, thus allowing, e.g. the treatment of cancer, but also for that region within PRDX4 when present on the cell surface of cancer cells, i.e., the region from amino acis 81 to 271 in human PRDX4 shown in SEQ ID NO: 81, which discriminates antibodies being able to detect PRDX4 when present on the cell surface of cancer cells. This is because, antibodies which bind to a region within, e.g. amino acids 1 to 46 fail to detect PRDX4 when present on the cell surface of cancer cells (see EP-A1 2546269, Example 8.2).

Within such a region, e.g. from amino acids 1 to 79, 37 to 79 or 81 to 271 shown in SEQ ID NO: 81 an antibody as described herein binds an epitope. Thus, when referring herein to “a region from amino acids 1 to 79 shown in SEQ ID NO: 81”, or “a region from amino acids 37 to 79 shown in SEQ ID NO: 81”, or “a region from amino acids 81 to 271 shown in SEQ ID NO: 81” it is preferably meant that an antibody as described herein binds an epitope within a region from amino acids 1 to 79 or 37 to 79 or 81 to 271 shown in SEQ ID NO: 81.

An epitope within a region from amino acids 1 to 79 shown in SEQ ID NO: 81 is preferred, more preferred is an epitope within a region from amino acids 37 to 79 shown in SEQ ID NO: 81, even more preferred is an epitope within a region from amino acids or 81 to 271 shown in SEQ ID NO: 81.

In one embodiment of the pharmaceutical composition of the present invention, said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16;
(i) an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(j) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

In one preferred embodiment of the pharmaceutical composition of the present invention, said antibody is
(a) an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2;
(b) an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4;
(c) an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6;
(d) an antibody comprising a heavy chain variable region having an amino acid sequence with at least 90% identity to the amino acid sequence shown in SEQ ID NO: 7 and a light chain variable region having an amino acid sequence with at least 90% identity to the amino acid sequence shown in SEQ ID NO: 8;
(e) an antibody comprising a heavy chain variable region having an amino acid sequence with at least 90% identity to the amino acid sequence shown in SEQ ID NO: 9 and a light chain variable region having an amino acid sequence with at least 90% identity to the amino acid sequence shown in SEQ ID NO: 10;
(f) an antibody comprising a heavy chain variable region having an amino acid sequence with at least 90% identity to the amino acid sequence shown in SEQ ID NO: 11 and a light chain variable region having an amino acid sequence with at least 90% identity to the amino acid sequence shown in SEQ ID NO: 12;
(g) an antibody comprising a heavy chain variable region having an amino acid sequence with at least 90% identity to the amino acid sequence shown in SEQ ID NO: 13 and a light chain variable region having an amino acid sequence with at least 90% identity to the amino acid sequence shown in SEQ ID NO: 14;
(h) an antibody comprising a heavy chain variable region having an amino acid sequence with at least 90% identity to the amino acid sequence shown in SEQ ID NO: 15 and a light chain variable region having an amino acid sequence with at least 90% identity to the amino acid sequence shown in SEQ ID NO: 16;
(i) an antibody comprising a heavy chain variable region having an amino acid NO: 17 and a light chain variable region having an amino acid sequence with at least 90% identity to the amino acid sequence shown in SEQ ID NO: 18;
(j) an antibody comprising a heavy chain variable region having an amino acid sequence with at least 90% identity to the amino acid sequence shown in SEQ ID NO: 19 and a light chain variable region having an amino acid sequence with at least 90% identity to the amino acid sequence shown in SEQ ID NO: 20; or
(k) an antibody which binds to the same epitope as that in the human PRDX4 protein to which any one of the antibodies (a) to (j) bind.

In one more preferred embodiment of the pharmaceutical composition of the present invention, said antibody is
(a) an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2;
(b) an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4;
(c) an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6;
(d) an antibody comprising a heavy chain variable region having an amino acid sequence with at least 95% identity to the amino acid sequence shown in SEQ ID NO: 7 and a light chain variable region having an amino acid sequence with at least 95% identity to the amino acid sequence shown in SEQ ID NO: 8;
(e) an antibody comprising a heavy chain variable region having an amino acid sequence with at least 95% identity to the amino acid sequence shown in SEQ ID NO: 9 and a light chain variable region having an amino acid sequence with at least 95% identity to the amino acid sequence shown in SEQ ID NO: 10;
(f) an antibody comprising a heavy chain variable region having an amino acid sequence with at least 95% identity to the amino acid sequence shown in SEQ ID NO: 11 and a light chain variable region having an amino acid sequence with at least 95% identity to the amino acid sequence shown in SEQ ID NO: 12;
(g) an antibody comprising a heavy chain variable region having an amino acid sequence with at least 95% identity to the amino acid sequence shown in SEQ ID NO: 13 and a light chain variable region having an amino acid sequence with at least 95% identity to the amino acid sequence shown in SEQ ID NO: 14;
(h) an antibody comprising a heavy chain variable region having an amino acid sequence with at least 95% identity to the amino acid sequence shown in SEQ ID NO: 15 and a light chain variable region having an amino acid sequence with at least 95% identity to the amino acid sequence shown in SEQ ID NO: 16;
(i) an antibody comprising a heavy chain variable region having an amino acid sequence with at least 95% identity to the amino acid sequence shown in SEQ ID NO: 17 and a light chain variable region having an amino acid sequence with at least 95% identity to the amino acid sequence shown in SEQ ID NO: 18;
(j) an antibody comprising a heavy chain variable region having an amino acid sequence with at least 95% identity to the amino acid sequence shown in SEQ ID NO: 19 and a light chain variable region having an amino acid sequence with at least 95% identity to the amino acid sequence shown in SEQ ID NO: 20; or
(k) an antibody which binds to the same epitope as that in the human PRDX4 protein to which any one of the antibodies (a) to (j) bind.

In one even more preferred embodiment of the pharmaceutical composition of the present invention, said antibody is
(a) an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2;
(b) an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4;
(c) an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6;
(d) an antibody comprising a heavy chain variable region having an amino acid sequence with at least 99% identity to the amino acid sequence shown in SEQ ID NO: 7 and a light chain variable region having an amino acid sequence with at least 99% identity to the amino acid sequence shown in SEQ ID NO: 8;
(e) an antibody comprising a heavy chain variable region having an amino acid NO: 9 and a light chain variable region having an amino acid sequence with at least 99% identity to the amino acid sequence shown in SEQ ID NO: 10;
(f) an antibody comprising a heavy chain variable region having an amino acid sequence with at least 99% identity to the amino acid sequence shown in SEQ ID NO: 11 and a light chain variable region having an amino acid sequence with at least 99% identity to the amino acid sequence shown in SEQ ID NO: 12;
(g) an antibody comprising a heavy chain variable region having an amino acid sequence with at least 99% identity to the amino acid sequence shown in SEQ ID NO: 13 and a light chain variable region having an amino acid sequence with at least 99% identity to the amino acid sequence shown in SEQ ID NO: 14;
(h) an antibody comprising a heavy chain variable region having an amino acid sequence with at least 99% identity to the amino acid sequence shown in SEQ ID NO: 15 and a light chain variable region having an amino acid sequence with at least 99% identity to the amino acid sequence shown in SEQ ID NO: 16;
(i) an antibody comprising a heavy chain variable region having an amino acid sequence with at least 99% identity to the amino acid sequence shown in SEQ ID NO: 17 and a light chain variable region having an amino acid sequence with at least 99% identity to the amino acid sequence shown in SEQ ID NO: 18;
(j) an antibody comprising a heavy chain variable region having an amino acid sequence with at least 99% identity to the amino acid sequence shown in SEQ ID NO: 19 and a light chain variable region having an amino acid sequence with at least 99% identity to the amino acid sequence shown in SEQ ID NO: 20; or
(k) an antibody which binds to the same epitope as that in the human PRDX4 protein to which any one of the antibodies (a) to (j) bind.

In one embodiment of the pharmaceutical composition of the present invention, said antibody is
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23, and
   a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
   a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
   a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
   a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
   a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
   a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
   a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62,
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
   a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68,
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
   a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74,
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
   a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80, or
(k) an antibody which binds to the same epitope as that in the human PRDX4 protein to which any one of the antibodies (a) to (j) bind.

The antibodies (a) to (k) as used in the pharmaceutical composition of the present invention and as described herein may contain constant regions. The constant regions used are not particularly limited, and any constant region may be used. Preferable examples of the constant regions used in the present invention can include human-derived constant regions. For example, a human IgG1-derived, human IgG2-derived, human IgG3-derived, or human IgG4-derived constant region can be used as a heavy chain constant region. Also, for example, human K chain-derived or human A chain-derived constant region can be used as a light chain constant region. The constant regions used in the present invention may be constant regions having a native sequence or may be modified constant regions having a sequence derived from the native sequence by the modification of one or more amino acids.

The antibodies (a) to (k) as used in the pharmaceutical composition of the present invention and as described herein may also contain FRs. The FRs used are not particularly limited, and any FR may be used as long as the resulting antibody maintains its binding activity to human PRDX4 present on the cell surface of a target cell. Preferable examples of the FRs used in the present invention can include human antibody-derived FRs. Since the technique of FR replacement with the antigen binding activity of an antibody maintained is known in the art, those skilled in the art can appropriately select FRs. The FRs used in the present invention may be FRs having a native sequence or may be FRs having a sequence derived from the native sequence by the modification of one or more amino acids.

Specifically, the present invention may also employ an antibody recognizing the same epitope as that recognized by any of these antibodies (see items (k) in the embodiments described above).

Whether or not an antibody shares an epitope with a certain antibody can be confirmed based on their competition for the same epitope. The competition between the antibodies is detected by cross-blocking assay or the like. The cross-blocking assay is preferably, for example, competitive ELISA assay.

Provided that a candidate antibody can bind to human PRDX4 present on the cell surface of a target cell by at least 80%, preferably at least 85%, more preferably at least 90%, even more preferably at least 95%, compared to the binding activity obtained in a control test performed in the absence of the candidate antibody, this candidate antibody is determined as an antibody that binds to substantially the same epitope as that to which the antibody of the present invention binds or to which the antibody used in the pharmaceutical composition binds.

In a further aspect, the present invention provides the pharmaceutical composition according to the present invention for use in a method for the treatment of cancer in a human subject. It is preferred for said embodiment, that said cancer is characterized by cells, wherein human PRDX4 is present on their cell surface. It is also preferred for said embodiment that said cancer is breast cancer, prostate cancer, lung cancer, colorectal cancer, ovarian cancer, oral cavity squamous carcinoma, hematologic malignancy tumor, or glioma.

The pharmaceutical composition according to the present invention can be administered by standard routes. These include, but are not limited to: oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, subcutaneous or intramuscular) routes.

The present invention also comprises a method of treating cancer, wherein the method comprises administering a therapeutically effective amount of the pharmaceutical composition according to the present invention to a human subject. It is preferred for said embodiment, that said cancer is characterized by cells, wherein human PRDX4 is present on their cell surface. It is also preferred for said embodiment that said cancer is breast cancer, prostate cancer, lung cancer, colorectal cancer, ovarian cancer, oral cavity squamous carcinoma, hematologic malignancy tumor, or glioma.

Further, the present invention comprises a method of treating cancer, wherein the method comprises administering a therapeutically effective amount of the antibody used in the pharmaceutical composition according to the present invention to a human subject. It is preferred for said embodiment, that said cancer is characterized by cells, wherein human PRDX4 is present on their cell surface. It is also preferred for said embodiment that said cancer is breast cancer, prostate cancer, lung cancer, colorectal cancer, ovarian cancer, oral cavity squamous carcinoma, hematologic malignancy tumor, or glioma.

The present invention also provides a method of treating cancer, wherein the method comprises administering a therapeutically effective amount of the antibody according to the present invention as described herein to a human subject. It is preferred for said embodiment, that said cancer is characterized by cells, wherein human PRDX4 is present on their cell surface. It is also preferred for said embodiment that said cancer is breast cancer, prostate cancer, lung cancer, colorectal cancer, ovarian cancer, oral cavity squamous carcinoma, hematologic malignancy tumor, or glioma.

The term “therapeutically effective amount” refers to an amount of the antibody or pharmaceutical composition according to the present invention or drug effective to “treat” cancer in the human subject. Specifically, in the case of cancer, the therapeutically effective amount of the antibody/pharmaceutical composition/drug can reduce the number of cancer cells; reduce the tumor size; inhibit or stop cancer cell infiltration into peripheral organs; inhibit and stop tumor metastasis; inhibit and stop tumor growth; relieve to some extent one or more of the symptoms associated with the cancer, or a combination of such effects on cancer cells. To the extent the antibody/pharmaceutical composition/drug prevents the growth and/or kills existing cancer cells, it can be referred to as cytostatic and/or cytotoxic.

The present invention also covers the use of the pharmaceutical composition according to the present invention for the manufacture of a medicament for the treatment of cancer.

In one embodiment, the present invention also covers the use of an antibody of the pharmaceutical composition according to the present invention and as described herein for the manufacture of a medicament for the treatment of cancer.

In one further embodiment, the present invention also covers the use of an antibody according to the present invention and as described herein for the manufacture of a medicament for the treatment of cancer.

Terms such as “treating” or “treatment” or “to treat” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention or with the pharmaceutical composition or antibody according to the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

The antibody to be administered to humans can also be converted to a genetically recombinant antibody that has been engineered artificially, for example, for the purpose of reducing heteroantigenicity in humans. The genetically recombinant antibody encompasses, for example, chimeric antibodies and humanized antibodies as defined herein. These engineered antibodies can be produced using a method known in the art.

In a further aspect, the present invention provides a method for determining whether a human subject may suffer from cancer, comprising determining in a sample obtained from said human subject whether PRDX4 is present on the cell surface of cancer cells comprised by said sample.

Said method for determining whether a human subject may suffer from cancer may comprise the following steps:
providing a sample obtained from said human subject,
determining in said sample obtained from said human subject whether PRDX4 is present on the cell surface of cancer cells comprised by said sample,
correlating the presence of PRDX4 on the cell surface of cancer cells comprised by said sample with determining whether the human subject may suffer from cancer.

A “subject” or “human subject” in the context of the present invention is a human being. For example, the subject may be a patient being suspected of having a disease or clinical condition associated with cancer or being diagnosed with such a disease or clinical condition.

Said “sample” obtained from said human subject is preferably selected from the group consisting of a blood sample, a serum sample, a plasma sample, a cerebrospinal fluid sample, a saliva sample, a solubilised tissue sample and an urine sample or an extract of any of the aforementioned samples. Preferably, the sample is a serum sample or a plasma sample. Most preferably, the sample is a serum sample for determining whether a human subject may suffer from cancer.

PRX4 can exist in monomeric or multimeric form. Thus, in a particular embodiment of the method according to the present invention, the PRDX4 may be determined as homomultimer, particularly a homodecamer or a homopentamer. In another particular embodiment, the presence or absence of a heteromultimer of PRDX4 on the cell surface of cells comprised by said sample may be determined. The multimer—be it homo- or heteromultimer—has preferably an apparent molecular weight in the range of from about 158 kDa to about 660 kDa, preferably 330 kDa +/−50 kDa as determined by size exclusion chromatography using a gel filtration column under non-denaturing conditions.

The step of “determining in said sample obtained from said human subject whether PRDX4 is present on the cell surface of cancer cells comprised by said sample” may comprise to determine the presence of PRDX4 or the fragments thereof by contacting the sample with at least one PRDX4 binder. The at least one binder may, for example, be the antibody according to the present invention. It is preferred that at least one binder is less than 20% cross-reactive with other proteins, particularly other peroxiredoxins such as PRDX1, PRDX2, PRDX3, PRDXS and PRDX6, more preferably less than 15%, more preferably less than 10%, more preferably less than 5%, even more preferably less than 3%, even more preferably less than 2% and even more preferably less than 1% cross reactive. In a particular embodiment, the at least one binder binds to an epitope contained in positions 1-79 of PRDX4 according to SEQ ID NO: 81. In another particular embodiment, the at least one binder binds to an epitope contained in positions 37-79 of PRDX4 according to SEQ ID NO: 81. In a more particular embodiment, the at least one binder binds to an epitope contained in positions 81-271 of PRDX4 according to SEQ ID NO: 81.

The present invention also provides an antibody which recognizes a region from amino acids 1 to 79, preferably from amino acids 37 to 79, more preferably from amino acids 81 to 271, in human PRDX4 shown in SEQ ID NO: 81. Such an antibody may be additionally defined by any one of the heavy chain variable regions and light chain variable regions as defined herein. Such an antibody may, alternatively be additionally defined by a heavy chain variable region comprising any of the heavy chain CDR1, heavy chain CDR2 and heavy chain CDR3 as defined herein and a light chain variable region comprising any of the light chain CDR1, light chain CDR2 and light chain CDR3 as defined herein.

The present invention also provides an antibody which binds to human PRDX4 present on the cell surface of a target cell, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16;
(i) an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(j) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

In one preferred embodiment of the antibody, which binds to human PRDX4 present on the cell surface of a target cell, said antibody is
(a) an antibody comprising a heavy chain variable region having an amino acid sequence with at least 90% identity to the amino acid sequence shown in SEQ ID NO: 1 and a light chain variable region having an amino acid sequence with at least 90% identity to the amino acid sequence shown in SEQ ID NO: 2;
(b) an antibody comprising a heavy chain variable region having an amino acid sequence with at least 90% identity to the amino acid sequence shown in SEQ ID NO: 3 and a light chain variable region having an amino acid sequence with at least 90% identity to the amino acid sequence shown in SEQ ID NO: 4;
(c) an antibody comprising a heavy chain variable region having an amino acid sequence with at least 90% identity to the amino acid sequence shown in SEQ ID NO: 5 and a light chain variable region having an amino acid sequence with at least 90% identity to the amino acid sequence shown in SEQ ID NO: 6;
(d) an antibody comprising a heavy chain variable region having an amino acid NO: 7 and a light chain variable region having an amino acid sequence with at least 90% identity to the amino acid sequence shown in SEQ ID NO: 8;
(e) an antibody comprising a heavy chain variable region having an amino acid sequence with at least 90% identity to the amino acid sequence shown in SEQ ID NO: 9 and a light chain variable region having an amino acid sequence with at least 90% identity to the amino acid sequence shown in SEQ ID NO: 10;
(f) an antibody comprising a heavy chain variable region having an amino acid sequence with at least 90% identity to the amino acid sequence shown in SEQ ID NO: 11 and a light chain variable region having an amino acid sequence with at least 90% identity to the amino acid sequence shown in SEQ ID NO: 12;
(g) an antibody comprising a heavy chain variable region having an amino acid sequence with at least 90% identity to the amino acid sequence shown in SEQ ID NO: 13 and a light chain variable region having an amino acid sequence with at least 90% identity to the amino acid sequence shown in SEQ ID NO: 14;
(h) an antibody comprising a heavy chain variable region having an amino acid sequence with at least 90% identity to the amino acid sequence shown in SEQ ID NO: 15 and a light chain variable region having an amino acid sequence with at least 90% identity to the amino acid sequence shown in SEQ ID NO: 16;
(i) an antibody comprising a heavy chain variable region having an amino acid sequence with at least 90% identity to the amino acid sequence shown in SEQ ID NO: 17 and a light chain variable region having an amino acid sequence with at least 90% identity to the amino acid sequence shown in SEQ ID NO: 18;
(j) an antibody comprising a heavy chain variable region having an amino acid sequence with at least 90% identity to the amino acid sequence shown in SEQ ID NO: 19 and a light chain variable region having an amino acid sequence with at least 90% identity to the amino acid sequence shown in SEQ ID NO: 20; or
(k) an antibody which binds to the same epitope as that in the human PRDX4 protein to which any one of the antibodies (a) to (j) bind.

In one more preferred embodiment of the antibody, which binds to human PRDX4 present on the cell surface of a target cell, said antibody is
(a) an antibody comprising a heavy chain variable region having an amino acid sequence with at least 95% identity to the amino acid sequence shown in SEQ ID NO: 1 and a light chain variable region having an amino acid sequence with at least 95% identity to the amino acid sequence shown in SEQ ID NO: 2;
(b) an antibody comprising a heavy chain variable region having an amino acid sequence with at least 95% identity to the amino acid sequence shown in SEQ ID NO: 3 and a light chain variable region having an amino acid sequence with at least 95% identity to the amino acid sequence shown in SEQ ID NO: 4;
(c) an antibody comprising a heavy chain variable region having an amino acid sequence with at least 95% identity to the amino acid sequence shown in SEQ ID NO: 5 and a light chain variable region having an amino acid sequence with at least 95% identity to the amino acid sequence shown in SEQ ID NO: 6;
(d) an antibody comprising a heavy chain variable region having an amino acid sequence with at least 95% identity to the amino acid sequence shown in SEQ ID NO: 7 and a light chain variable region having an amino acid sequence with at least 95% identity to the amino acid sequence shown in SEQ ID NO: 8;
(e) an antibody comprising a heavy chain variable region having an amino acid sequence with at least 95% identity to the amino acid sequence shown in SEQ ID NO: 9 and a light chain variable region having an amino acid sequence with at least 95% identity to the amino acid sequence shown in SEQ ID NO: 10;
(f) an antibody comprising a heavy chain variable region having an amino acid sequence with at least 95% identity to the amino acid sequence shown in SEQ ID NO: 11 and a light chain variable region having an amino acid sequence with at least 95% identity to the amino acid sequence shown in SEQ ID NO: 12;
(g) an antibody comprising a heavy chain variable region having an amino acid sequence with at least 95% identity to the amino acid sequence shown in SEQ ID NO: 13 and a light chain variable region having an amino acid sequence with at least 95% identity to the amino acid sequence shown in SEQ ID NO: 14;
(h) an antibody comprising a heavy chain variable region having an amino acid sequence with at least 95% identity to the amino acid sequence shown in SEQ ID NO: 15 and a light chain variable region having an amino acid sequence with at least 95% identity to the amino acid sequence shown in SEQ ID NO: 16;
(i) an antibody comprising a heavy chain variable region having an amino acid sequence with at least 95% identity to the amino acid sequence shown in SEQ ID NO: 17 and a light chain variable region having an amino acid sequence with at least 95% identity to the amino acid sequence shown in SEQ ID NO: 18;
(j) an antibody comprising a heavy chain variable region having an amino acid sequence with at least 95% identity to the amino acid sequence shown in SEQ ID NO: 19 and a light chain variable region having an amino acid sequence with at least 95% identity to the amino acid sequence shown in SEQ ID NO: 20; or
(k) an antibody which binds to the same epitope as that in the human PRDX4 protein to which any one of the antibodies (a) to (j) bind.

In one even more preferred embodiment of the antibody, which binds to human PRDX4 present on the cell surface of a target cell, said antibody is
(a) an antibody comprising a heavy chain variable region having an amino acid sequence with at least 99% identity to the amino acid sequence shown in SEQ ID NO: 1 and a light chain variable region having an amino acid sequence with at least 99% identity to the amino acid sequence shown in SEQ ID NO: 2;
(b) an antibody comprising a heavy chain variable region having an amino acid sequence with at least 99% identity to the amino acid sequence shown in SEQ ID NO: 3 and a light chain variable region having an amino acid sequence with at least 99% identity to the amino acid sequence shown in SEQ ID NO: 4;
(c) an antibody comprising a heavy chain variable region having an amino acid sequence with at least 99% identity to the amino acid sequence shown in SEQ ID NO: 5 and a light chain variable region having an amino acid sequence with at least 99% identity to the amino acid sequence shown in SEQ ID NO: 6;
(d) an antibody comprising a heavy chain variable region having an amino acid sequence with at least 99% identity to the amino acid sequence shown in SEQ ID NO: 7 and a light chain variable region having an amino acid sequence with at least 99% identity to the amino acid sequence shown in SEQ ID NO: 8;
(e) an antibody comprising a heavy chain variable region having an amino acid sequence with at least 99% identity to the amino acid sequence shown in SEQ ID NO: 9 and a light chain variable region having an amino acid sequence with at least 99% identity to the amino acid sequence shown in SEQ ID NO: 10;
(f) an antibody comprising a heavy chain variable region having an amino acid sequence with at least 99% identity to the amino acid sequence shown in SEQ ID NO: 11 and a light chain variable region having an amino acid sequence with at least 99% identity to the amino acid sequence shown in SEQ ID NO: 12;
(g) an antibody comprising a heavy chain variable region having an amino acid sequence with at least 99% identity to the amino acid sequence shown in SEQ ID NO: 13 and a light chain variable region having an amino acid sequence with at least 99% identity to the amino acid sequence shown in SEQ ID NO: 14;
(h) an antibody comprising a heavy chain variable region having an amino acid sequence with at least 99% identity to the amino acid sequence shown in SEQ ID NO: 15 and a light chain variable region having an amino acid sequence with at least 99% identity to the amino acid sequence shown in SEQ ID NO: 16;
(i) an antibody comprising a heavy chain variable region having an amino acid sequence with at least 99% identity to the amino acid sequence shown in SEQ ID NO: 17 and a light chain variable region having an amino acid sequence with at least 99% identity to the amino acid sequence shown in SEQ ID NO: 18;
(j) an antibody comprising a heavy chain variable region having an amino acid sequence with at least 99% identity to the amino acid sequence shown in SEQ ID NO: 19 and a light chain variable region having an amino acid sequence with at least 99% identity to the amino acid sequence shown in SEQ ID NO: 20; or
(k) an antibody which binds to the same epitope as that in the human PRDX4 protein to which any one of the antibodies (a) to (j) bind.

In one further embodiment, the present invention provides an antibody which binds to human PRDX4 present on the cell surface of a target cell, which comprises
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23 and
   a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
   a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
   a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
   a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
   a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
   a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
   a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62;
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
   a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68;
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
   a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74;
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
   a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80; or
(k) an antibody which binds to the same epitope as that in the human PRDX4 protein to which any one of the antibodies (a) to (j) bind.

Any of the above described antibodies may preferably bind a region from amino acids 81 to 271, in human PRDX4 shown in SEQ ID NO: 81.

The present invention also provides the use of the antibody of the present invention in the manufacture of a medicament for the treatment of cancer.

The invention additionally provides the antibody of the present invention for use in a method of treating cancer.

In some embodiments, the antibody of the present invention may contain human Fc regions that are modified to enhance effector function, for example, antigen-dependent cell-mediated cytotoxicity (ADCC) and/or complement dependent cytotoxicity (CDC). This can be achieved by introducing one or more amino acid substitutions in a Fc region of the antibody. For example, cysteine residue(s) can be introduced in the Fc region to allow interchain disulfide bond formation in this region to improve complement-mediated cell killing and antibody-dependent cellular cytotoxicity (ADCC). Homodimeric antibodies with enhanced anti-tumor activity can also be prepared using heterobifunctional cross-linkers as known to the person skilled in the art. Alternatively, an antibody may be engineered which has dual Fc regions.

Whether an antibody to be tested recognizes the same epitope as that recognized by a certain antibody as described above under (k), i.e., these antibodies share the epitope, can be confirmed based on their competition for the same epitope. The competition between the antibodies is detected by a cross-blocking assay or the like. For example, competitive ELISA assay is a preferably used cross-blocking assay. Specifically, in the cross-blocking assay, PRDX4 proteins coated on the wells of a microtiter plate may be preincubated in the presence or absence of a candidate competing antibody, and the antibody of the present invention is then added to the wells. The amount of the antibody of the present invention bound to the PRDX4 protein in the well indirectly correlates with the binding ability of the candidate competing antibody (antibody to be tested) that competes therewith for the binding to the same epitope. Specifically, the larger affinity the antibody to be tested has for the same epitope, the smaller amount of the antibody of the present invention is bound to the PRDX4 protein-coated well, while the larger amount of the antibody to be tested is bound to the PRDX4 protein-coated well.

The amount of the antibody bound to the well can be measured easily by labeling the antibody in advance. For example, a biotin-labeled antibody can be measured by use of an avidin-peroxidase conjugate and an appropriate substrate. The cross-blocking assay using enzyme (e.g., peroxidase) labeling is particularly referred to as competitive ELISA assay. The antibody can be labeled with other detectable or measurable labeling substances. Specifically, radio-labeling or fluorescent labeling or the like is known in the art.

Furthermore, the antibodies (a) to (j) as described above may have substitution, deletion, addition and/or insertion of one or more amino acids in their CDR sequences as long as the resulting antibodies are functionally equivalent to the antibodies (a) to (j). The term “functionally equivalent” refers to being comparable in avidity for PRDX4 and cytotoxicity. The term “equivalent” refers to having at least 50%, preferably having at least 60%, more preferably having at least 70%, more preferably having at least 80%, even more preferably at least 90%, even more preferably at least 95% and even more preferably at least 99% activity, compared with the antibodies (a) to (j). The upper limit of the activity is not particularly limited and may be higher than that of the antibodies (a) to (j). The avidity or cytotoxicity can be assayed by a method generally known by those skilled in the art.

The antibody according to the present invention may be capable of binding to a PRDX4 polypeptide extracellularly. In other words, an antibody as described herein may be capable of binding to PRDX4 when it is outside the cell, which also comprises PRDX4 to be specifically on the surface of the cell.

The description of the sequences, shown in the sequence listing and as used in the context of the present invention, is as follows:

SEQ ID NO: 1 shows the amino acid sequence of the VH-region of antibody 2C1.

SEQ ID NO: 2 shows the amino acid sequence of the VL-region of antibody 2C1.

SEQ ID NO: 3 shows the amino acid sequence of the VH-region of antibody 5H5.

SEQ ID NO: 4 shows the amino acid sequence of the VL-region of antibody 5H5.

SEQ ID NO: 5 shows the amino acid sequence of the VH-region of antibody 10G7.

SEQ ID NO: 6 shows the amino acid sequence of the VL-region of antibody 10G7.

SEQ ID NO: 7 shows the amino acid sequence of the VH-region of antibody 11-F2.

SEQ ID NO: 8 shows the amino acid sequence of the VL-region of antibody 11-F2.

SEQ ID NO: 9 shows the amino acid sequence of the VH-region of antibody 13B2.

SEQ ID NO: 10 shows the amino acid sequence of the VL-region of antibody 13B2.

SEQ ID NO: 11 shows the amino acid sequence of the VH-region of antibody 15-F11.

SEQ ID NO: 12 shows the amino acid sequence of the VL-region of antibody 15-F11.

SEQ ID NO: 13 shows the amino acid sequence of the VH-region of antibody 17A10.

SEQ ID NO: 14 shows the amino acid sequence of the VL-region of antibody 17A10.

SEQ ID NO: 15 shows the amino acid sequence of the VH-region of antibody 18G8.

SEQ ID NO: 16 shows the amino acid sequence of the VL-region of antibody 18G8.

SEQ ID NO: 17 shows the amino acid sequence of the VH-region of antibody 19H9.

SEQ ID NO: 18 shows the amino acid sequence of the VL-region of antibody 19H9.

SEQ ID NO: 19 shows the amino acid sequence of the VH-region of antibody 24B7.

SEQ ID NO: 20 shows the amino acid sequence of the VL-region of antibody 24B7.

SEQ ID NO: 21 shows the amino acid sequence of the VH-CDR1 of antibody 2C1.

SEQ ID NO: 22 shows the amino acid sequence of the VH-CDR2 of antibody 2C1.

SEQ ID NO: 23 shows the amino acid sequence of the VH-CDR3 of antibody 2C1.

SEQ ID NO: 24 shows the amino acid sequence of the VL-CDR1 of antibody 2C1.

SEQ ID NO: 25 shows the amino acid sequence of the VL-CDR2 of antibody 2C1.

SEQ ID NO: 26 shows the amino acid sequence of the VL-CDR3 of antibody 2C1.

SEQ ID NO: 27 shows the amino acid sequence of the VH-CDR1 of antibody 5H5.

SEQ ID NO: 28 shows the amino acid sequence of the VH-CDR2 of antibody 5H5.

SEQ ID NO: 29 shows the amino acid sequence of the VH-CDR3 of antibody 5H5.

SEQ ID NO: 30 shows the amino acid sequence of the VL-CDR1 of antibody 5H5.

SEQ ID NO: 31 shows the amino acid sequence of the VL-CDR2 of antibody 5H5.

SEQ ID NO: 32 shows the amino acid sequence of the VH-CDR3 of antibody 5H5.

SEQ ID NO: 33 shows the amino acid sequence of the VH-CDR1 of antibody 10G7.

SEQ ID NO: 34 shows the amino acid sequence of the VH-CDR2 of antibody 10G7.

SEQ ID NO: 35 shows the amino acid sequence of the VH-CDR3 of antibody 10G7.

SEQ ID NO: 36 shows the amino acid sequence of the VL-CDR1 of antibody 10G7.

SEQ ID NO: 37 shows the amino acid sequence of the VL-CDR2 of antibody 10G7.

SEQ ID NO: 38 shows the amino acid sequence of the VL-CDR3 of antibody 10G7.

SEQ ID NO: 39 shows the amino acid sequence of the VH-CDR1 of antibody 11-F2.

SEQ ID NO: 40 shows the amino acid sequence of the VH-CDR2 of antibody 11-F2.

SEQ ID NO: 41 shows the amino acid sequence of the VH-CDR3 of antibody 11-F2.

SEQ ID NO: 42 shows the amino acid sequence of the VL-CDR1 of antibody 11-F2.

SEQ ID NO: 43 shows the amino acid sequence of the VL-CDR2 of antibody 11-F2.

SEQ ID NO: 44 shows the amino acid sequence of the VL-CDR3 of antibody 11-F2.

SEQ ID NO: 45 shows the amino acid sequence of the VH-CDR1 of antibody 13B2.

SEQ ID NO: 46 shows the amino acid sequence of the VH-CDR2 of antibody 13B2.

SEQ ID NO: 47 shows the amino acid sequence of the VH-CDR3 of antibody 13B2.

SEQ ID NO: 48 shows the amino acid sequence of the VL-CDR1 of antibody 13B2.

SEQ ID NO: 49 shows the amino acid sequence of the VL-CDR2 of antibody 13B2.

SEQ ID NO: 50 shows the amino acid sequence of the VL-CDR3 of antibody 13B2.

SEQ ID NO: 51 shows the amino acid sequence of the VH-CDR1 of antibody 15-F11.

SEQ ID NO: 52 shows the amino acid sequence of the VH-CDR2 of antibody 15-F11.

SEQ ID NO: 53 shows the amino acid sequence of the VH-CDR3 of antibody 15-F11.

SEQ ID NO: 54 shows the amino acid sequence of the VL-CDR1 of antibody 15-F11.

SEQ ID NO: 55 shows the amino acid sequence of the VL-CDR2 of antibody 15-F11

SEQ ID NO: 56 shows the amino acid sequence of the VL-CDR3 of antibody 15-F11.

SEQ ID NO: 57 shows the amino acid sequence of the VH-CDR1 of antibody 17A10.

SEQ ID NO: 58 shows the amino acid sequence of the VH-CDR2 of antibody 17A10.

SEQ ID NO: 59 shows the amino acid sequence of the VH-CDR3 of antibody 17A10.

SEQ ID NO: 60 shows the amino acid sequence of the VL-CDR1 of antibody 17A10.

SEQ ID NO: 61 shows the amino acid sequence of the VL-CDR2 of antibody 17A10.

SEQ ID NO: 62 shows the amino acid sequence of the VL-CDR3 of antibody 17A10.

SEQ ID NO: 63 shows the amino acid sequence of the VH-CDR1 of antibody 18G8.

SEQ ID NO: 64 shows the amino acid sequence of the VH-CDR2 of antibody 18G8.

SEQ ID NO: 65 shows the amino acid sequence of the VH-CDR3 of antibody 18G8.

SEQ ID NO: 66 shows the amino acid sequence of the VL-CDR1 of antibody 18G8.

SEQ ID NO: 67 shows the amino acid sequence of the VL-CDR2 of antibody 18G8.

SEQ ID NO: 68 shows the amino acid sequence of the VL-CDR3 of antibody 18G8.

SEQ ID NO: 69 shows the amino acid sequence of the VH-CDR1 of antibody 19G9.

SEQ ID NO: 70 shows the amino acid sequence of the VH-CDR2 of antibody 19G9.

SEQ ID NO: 71 shows the amino acid sequence of the VH-CDR3 of antibody 19G9.

SEQ ID NO: 72 shows the amino acid sequence of the VL-CDR1 of antibody 19G9.

SEQ ID NO: 73 shows the amino acid sequence of the VL-CDR2 of antibody 19G9.

SEQ ID NO: 74 shows the amino acid sequence of the VL-CDR3 of antibody 19G9.

SEQ ID NO: 75 shows the amino acid sequence of the VH-CDR1 of antibody 24B7.

SEQ ID NO: 76 shows the amino acid sequence of the VH-CDR2 of antibody 24B7.

SEQ ID NO: 77 shows the amino acid sequence of the VH-CDR3 of antibody 24B7.

SEQ ID NO: 78 shows the amino acid sequence of the VL-CDR1 of antibody 24B7.

SEQ ID NO: 79 shows the amino acid sequence of the VL-CDR2 of antibody 24B7.

SEQ ID NO: 80 shows the amino acid sequence of the VL-CDR3 of antibody 24B7.

SEQ ID NO: 81 shows the amino acid sequence of human PRDX4.

SEQ ID NO: 82 shows the amino acid sequence from positions 38 to 46 within SEQ ID NO: 81.

An overview of the SEQ ID NOs and detailed sequences, as used in the context of the present invention, is given in the following Table 1 (in case of conflict between the sequences shown in Table 1 and the sequences of the sequence listing which has to be filed for formal reasons, the sequences of Table 1 supercede the sequences of the sequence listing):

TABLE 1

| SEQ | | Sequence |
|---|---|---|
| ID | | (CDRs are marked in bold in the order CDR |
| NO: | Designation | 1, 2, 3 starting from left upper line) |
| 1 | 2C1 VH | QVQLQQSGAELVKPGTSVKLSCKASGYTFTSNHMNWIKQTTGQGLEWIGIINSGNGGTKYNVKFKGKAT |
| | | LTVDKSSSTAFMQLSSLTPEDSAVYYCARGNPASFDYWGQGVMVTVSS |
| 2 | 2C1 VL | DIQMTQSPSFLSASVGDRVTINCKASQNINKYLNWYQHKLGEAPKRLIYNTDNLQTGIPSRFSGSGSGTD |
| | | YTLTISGLQPEDFATYFCLQHSNFPFTFGSGTKLEIARAD |
| 3 | 5H5 VH | QVQLQQSGAELVRPGTSVKLSCKASGYTFTSNHMHWIKETTGQGLEWIGIINPGNGGSRFNVKFKGKAT |
| | | LTVDKSSTTAFMQLSSLTPEDSAVYYCARGNPASFDYWGQGVMVTVSS |
| 4 | 5H5 VL | DIQMTQSPSFLSASVGDRVTINCRASENIYKYLNWYQQKFGEAPKRLIYNTNTLETGIPSRFSGSGSGTD |
| | | FTLTISSLQPEDFATYFCLQHSNFPFTFGSGTKLEIKRA |
| 5 | 10G7 VH | QVQLQQSGAELVKPGTSVKLSCKASGYTFTSNHMNWIKQTTGQGLEWIGIINPVNGGTRYNVKFKGKAT |
| | | LTVDKSSSTAFMQLSSLTPEDSAVYYCARGNPASFDYWGQGVMVTVSS |
| 6 | 10G7 VL | DIQMTQSPSFLSASVGDRVTINCKASQNIHKYLNWYQHKLGEAPKRLIYNTNNLQTDIPSNFSGSGSGTD |
| | | YTLTISSLQPEDFATYFCLQHSSFPFTFGSGTKLEIKRA |
| 7 | 11-F2 VH | QVQLQQSGAELVKPGTSVKLSCKASGYTFSTHHMHWIKETTGQGLEWIGIINPGNGGTKYNVKFKGKAT |
| | | LTVDSSSSTAFMQLGSLTPEDSAVYYCARGNPASFDYWGQGVMVTVSS |
| 8 | 11-F2 VL | DIQMTQSPSFLSASVGDRVTINCKASQNIYKYLNWYQQKLGEAPKRLIYNTNNLETDIPSRFSGSGSGTD |
| | | YTFTISSLQPEDFATYFCLQHSNFPFTFGSGTKLEIKRA |
| 9 | 13B2 VH | QVQLQQSGAELVRPGTSVKLSCKASGYTFTSNHMNWIKETTGQGLEWIGIINPGNGGSRFNVKFKGKAT |
| | | LTVDKSSTTAFMQLSSLTPEDSAVYYCARGNPASFDYWGQGVMVTVSS |
| 10 | 13B2 VL | DIQMTQSPSFLSASVGDRVTINCRASENIYKYLNWYQQKLGEAPKRLIYNTNTLETGIPSRFSGSGSGTD |
| | | FTLTISSLOPEDFATYFCLQHSNFPFTFGSGTKLEIKRA |
| 11 | 15-F11 VH | QVQLQQSGAELVRPGTSVKLSCKASGYTFTSNHMHWIKETTGQGLEWIGIINPGNGGSRFNVKFKGKAT |
| | | LTVDKSSTTAFMQLSSLTPEDSAVYYCARGNPASFDYWGQGVMVTVSS |
| 12 | 15-F11 VL | DIQMTQSPSFLSASVGDRVTFNCRASENIYNYLNWYQRKFGEAPKRLISNTNTLETGIPSRFSGSGSGTD |
| | | FTLTISSLQPEDFATYFCLQHSNFPFTFGSGTKLEIKRA |
| 13 | 17A10 VH | QVQLQQSGAELVRPGTSVKLSCKASGYTFTSNHMHWIKETTGQGLEWIGIINPGNGGSRFNVKFKGKAT |
| | | LTVDKSYTTAFMQLSSLTPEDSAVYYCARGNPASFDYWGRGVMVTVSS |
| 14 | 17A10 VL | DIQMTQSPSFLSASVGDRVTINCRASENIYNYLNWYQQKFGEAPKRLIYNTNTLETGIPSRFSGSGSGTD |
| | | FTLTISSLQPEDFATYFCLQHSNFPFTFGSGTKLEIKRA |
| 15 | 18G8 VH | QVQLQQSGAELVEPGTSVRLSCKASGYTFTNNHIHWIKQATGQALEWIGIINSGNGGTKYDVKFKGKATL |
| | | TVDKSSNTAFMQLSSLTPEDSAVYYCARGNPASFDYWGQGVLVTVSS |
| 16 | 18G8 VL | DIQMTQSPSFLSASVGDRLTINCKASQNIYKYLNWYQQKLGEAPKRLIYDTNNLQTGIPSRFSGSGSGTD |
| | | YTLTISSLQPEDFATYFCLQHSNFPFTFGSGTKLEIKRA |
| 17 | 19H9 VH | QVQLQQSGVELVRPGTSVRLSCKASGYTFTSNHMHWIKETTGQGLEWIGIINPGNGGSRFNVKFKGKAT |
| | | LTVDKSSTTAFMQLNSLTPEDSAVYYCARGNPASFDYWGQGVMVTVSS |
| 18 | 19H9 VL | DIQMTQSPSFLSASVGDRVTINCRASENIYNYLNWYQQKFGEGPKRLIYNTNTLETGIPSRFSGSGSGTD |
| | | FTLTINSLQPEDFATYFCLQHSNFPFTFGSGTKLEIKRA |
| 19 | 24B7 VH | QVQLQQSGAELVRPGTSVKLSCKASGYTFTSNHMHWIKETTGQGLEWIGIINPGNGGSRFNVKFKGKAT |
| | | LTVDKSSTTAFMQLSSLTPEDSAVYYCARGNPASFDYWGRGVMVTVSS |
| 20 | 24B7 VL | DIQMTQSPSFLSASVGDRVTINCRASENIYNYLNWYQQKFGEAPKRLIYNTNTLETGIPSRFSGSGSGTD |
| | | FTLTISSLQPEDFATYFCLQHSNFPFTFGSGTKLEIKRA |
| 21 | 2C1 VH-CDR1 | TSNHMN |
| 22 | 2C1 VH-CDR2 | IINSGNGGTKYNVKFKG |
| 23 | 2C1 VH-CDR3 | GNPASFDY |
| 24 | 2C1 VL-CDR1 | KASQNINKYLN |
| 25 | 2C1 VL-CDR2 | NTDNLQT |
| 26 | 2C1 VL-CDR3 | LQHSNFPFT |
| 27 | 5H5 VH-CDR1 | TSNHMH |
| 28 | 5H5 VH-CDR2 | IINPGNGGSRFNVKFKG |
| 29 | 5H5 VH-CDR3 | GNPASFDY |
| 30 | 5H5 VL-CDR1 | RASENIYKYLN |
| 31 | 5H5 VL-CDR2 | NTNTLETG |
| 32 | 5H5 VL-CDR3 | LQHSNFPFT |
| 33 | 10G7 VH-CDR1 | TSNHMN |
| 34 | 10G7 VH-CDR2 | IINPVNGGTRYNVKFKG |
| 35 | 10G7 VH-CDR3 | GNPASFDY |
| 36 | 10G7 VL-CDR1 | KASQNIHKYLN |
| 37 | 10G7 VL-CDR2 | NTNNLQT |
| 38 | 10G7 VL-CDR3 | LQHSSFPFT |
| 39 | 11-F2 VH-CDR1 | STHHMH |
| 40 | 11-F2 VH-CDR2 | IINPGNGGTKYNVKFKG |
| 41 | 11-F2 VH-CDR3 | GNPASFDY |
| 42 | 11-F2 VL-CDR1 | KASQNIYKYLN |
| 43 | 11-F2 VL-CDR2 | YNTNNLETD |
| 44 | 11-F2 VL-CDR3 | LQHSNFPFT |
| 45 | 13B2 VH-CDR1 | TSNHMN |
| 46 | 13B2 VH-CDR2 | IINPGNGGSRFNVKFKG |
| 47 | 13B2 VH-CDR3 | GNPASFDY |
| 48 | 13B2 VL-CDR1 | RASENIYKYLN |
| 49 | 13B2 VL-CDR2 | YNTNTLETG |
| 50 | 13B2 VL-CDR3 | LQHSNFPFT |
| 51 | 15-F11 VH-CDR1 | TSNHMH |
| 52 | 15-F11 VH-CDR2 | IINPGNGGSRFNVKFKG |
| 53 | 15-F11 VH-CDR3 | GNPASFDY |
| 54 | 15-F11 VL-CDR1 | RASENIYNYLN |
| 55 | 15-F11 VL-CDR2 | SNTNTLETG |
| 56 | 15-F11 VL-CDR3 | LQHSNFPFT |
| 57 | 17A10 VH-CDR1 | TSNHMH |
| 58 | 17A10 VH-CDR2 | IINPGNGGSRFNVKFKG |
| 59 | 17A10 VH-CDR3 | GNPASFDY |
| 60 | 17A10 VL-CDR1 | RASENIYNYLN |
| 61 | 17A10 VL-CDR2 | YNTNTLETG |
| 62 | 17A10 VL-CDR3 | LQHSNFPFT |
| 63 | 18G8 VH-CDR1 | TNNHIH |
| 64 | 18G8 VH-CDR2 | IINSGNGGTKYDVKFKG |
| 65 | 18G8 VH-CDR3 | GNPASFDY |
| 66 | 18G8 VL-CDR1 | KASQNIYKYLN |
| 67 | 18G8 VL-CDR2 | DTNNLQTG |
| 68 | 18G8 VL-CDR3 | LQHSNFPFT |
| 69 | 19H9 VH-CDR1 | TSNHMH |
| 70 | 19H9 VH-CDR2 | IINPGNGGSRFNVKFKG |
| 71 | 19H9 VH-CDR3 | GNPASFDY |
| 72 | 19H9 VL-CDR1 | RASENIYNYLN |
| 73 | 19H9 VL-CDR2 | NTNTLETG |
| 74 | 19H9 VL-CDR3 | LQHSNFPFT |
| 75 | 24B7 VH-CDR1 | TSNHMH |
| 76 | 24B7 VH-CDR2 | IINPGNGGSRFNVKFKG |
| 77 | 24B7 VH-CDR3 | GNPASFDY |
| 78 | 24B7 VL-CDR1 | RASENIYNYLN |
| 79 | 24B7 VL-CDR2 | NTNTLETG |
| 80 | 24B7 VL-CDR3 | LQHSNFPFT |
| 81 | Human PRDX4 | MEALPLLAATTPDHGRHRRLLLLPLLLFLLPAGAVQGWETEERPRTREEECHFYAGGQVYPGEASRVSV |
| | | ADHSLHLSKAKISKPAPYWEGTAVIDGEFKELKLTDYRGKYLVFFFYPLDFTFVCPTEIIAFGDRLEEFRSIN |
| | | TEVVACSVDSQFTHLAWINTPRRQGGLGPIRIPLLSDLTHQISKDYGVYLEDSGHTLRGLFIIDDKGILRQIT |
| | | LNDLPVGRSVDETLRLVQAFQYTDKHGEVCPAGWKPGSETIIPDPAGKLKYFDKLN |
| 82 | Human PRDX4 | WETEERPRT |

The following abbreviations were used in the context of the present invention: VH=variable heavy chain; VH=variable heavy chain; VL=variable light chain; CDR=complementarity determining region; VH-CDR=CDR of a variable heavy region; VL-CDR=CDR of a variable light region; CDRs can be determined by, e.g., http://abysis.org/abysis/.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or”, wherever used herein, includes the meaning of “and”, “or” and “all or any other combination of the elements connected by said term”.

The term “less than” or in turn “more than” does not include the concrete number.

For example, “less than 20” means less than the number indicated. Similarly, “more than” or “greater than” means more than or greater than the indicated number, e.g. “more than 80%” means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes, when used herein, with the term “having”. When used herein, “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES

### Materials and Methods:

### Cells and Culture Conditions:

Cells were cultured in DMEM/F12 medium (Gibco BRL, Karlsruhe, Germany), supplemented with 8% v/v fetal bovine serum, 1% v/v L-Glutamine and 1% v/v penicillin-streptomycin). All cells were incubated in a humidified CO₂ incubator at 37° C., 18.3% O₂ and 5% CO₂. Hypoxic cells were incubated at 1% O₂.

### Transfection of 293 Cells:

293 cells were transfected with expression plasmids, coding for human PRDX or mutants thereof using Lipofectamine™ 2000 transfection reagent (ThermoFisher Scientific, Menzel, Germany) according to manufacturer's instructions. PRDX4 specific siRNAs were transfected using Lipofectamine. Where appropriate, cells were selected for positive transfected cells with 80 μg/ml Hygromycin C and further cultured in normal growth medium supplemented with 80 μg/ml Hygromycin C.

Isolation of Primary Peripheral Blood Mononuclear Cells (PBMCs) from Fresh Blood:

30 ml fresh heparinized blood from healthy donors were diluted with PBS and loaded carefully on top of a layer of 10 ml human Pancoll (Pan Biotech, Aidenbach, Germany). The tubes were centrifuged for 25 min at 800×g. After that, PBMCs were carefully collected from the intermediate layer between Pancoll and plasma and washed three times in PBS. Cells were used fresh for experiments or immediately frozen and stored at −80° C. for later experiments.

### Flow Cytometry:

For flow cytometric analysis of PRDX4 expression, cells were stained with 11F2 or any other PRDX4-specific antibody in FACS-buffer (PBS+2% FSC) or with an isotype control antibody for 20 min followed by staining with an anti-rat-Alexa Fluor® 647 secondary antibody (Jackson Immuno Research). All stainings were performed on ice.

### Western Blotting Analysis:

For analysis of PRDX4 expression by western blotting, whole cell lysates were prepared. Adherent cells were washed with PBS, detached by trypsin and pelleted. The cell pellet was washed in PBS. After that, cells were resuspended in RIPA buffer with protease inhibitors (Roche Diagnostics) and incubated on ice for 20 min. Cell fragments were centrifuged for 20 min at 4° C. at max speed. The supernatant was transferred to a new tube and the protein content was determined by Bradford assay (Bio-Rad). 20 μg of cell lysate were used for SDS-PAGE. Gels were electroblotted on a nitrocellulose membrane and blocked with 5% milk in TBST. Following primary antibodies were used for Western blotting: anti-PRDX4 (Thermo Scientific), anti-GFP (kind gift of the core facility ‘monoclonal antibodies’, Helmholtz Center Munich), All primary antibodies were incubated at 4° C. over night. The membrane was washed and incubated with HRP-conjugated secondary antibodies at room temperature for 2 h. The following secondary antibodies were used: anti-rat IgG-HRP (Cell Signaling), anti-mouse IgG-HRP (Cell Signaling Technology, Frankfurt a. Main, Germany) and anti-rabbit IgG-HRP (Cell Signaling). Detection of proteins was performed with the ECL system (GE Healthcare, Freiburg, Germany).

### Immunoprecipitation:

Immunoprecipitation was performed using CNBr beads (CNBr-activated Sepharose 4 Fast Flow, GE Healthcare). 1 g beads were solved in 10 ml 1 mM HCl and incubated at room temperature for 20 min. After that beads were spin down for 1 min at 3000×g and washed 15×. Then, a subclass specific mouse anti-rat IgG2a antibody was coupled to the beads (2 mg antibody in coupling buffer 0.3 M NaHCO₃, 1.5 M NaCl, pH 8.3) for 1 h at room temperature. After that, beads were washed in coupling buffer and all remaining binding sites blocked with 1 M ethanolamine (1 M) for 2 h at room temperature. After washing in wash buffer (0.1 M Tris/HCl, 0.5 M NaCl, pH 4) and in NaOAc buffer (0.1 M NaOAc, 0.5 M NaCl) coupled beads were resuspended in PBS and used for coupling with 22E6. Therefore, 500 μl 22E6 hybridoma supernatant was incubated with 60 μl anti-subclass specific beads over night at 4° C.

Beads were then washed in PBS and incubated with 2 mg cell lysate or 0.75 μg sCD73 (human CD73/NTSE protein, his tagged, Sino Biologicals) at 4° C. over night. After that, beads were washed in RIPA buffer with protease inhibitors, PBS+(PBS, 0.5% N-laurylsarcosine, 0.1% SDS) and in PBS. The supernatant was discarded, beads resuspended in 3× Laemmli buffer and incubated at room temperature for 3 min. After centrifugation for 5 min at 1,000× g, the supernatant was used for western blot analysis.

### EXAMPLES

### Example 1

### PRDX4 is a Hypoxia-Induced Protein on the Surface of Human Cancer Cells

A549 lung cancer cells and ES-2 ovarian cancer cells were cultured in standard cell culture medium under normoxic (approx. 18% O₂) or hypoxic (1% O₂) conditions. The expression of surface PRDX4 was measured by flow cytometry using the antibody 11F2 as a primary antibody and an Alexa647-labelled goat-anti-rat IgG as a secondary antibody. The mean fluorescence intensities from Figure la) were measured (see FIG. 2b)). Given are the mean values and standard deviations from three independent experiments. No expression was observed on primary PBMCs, hepatocytes and cardiac fibroblasts (data not shown).

### Example 2

PRDX4 was expressed on various human cancer cell lines of different origin and on cancer cells isolated from primary ascites from patients with ovarian cancer (see FIG. 2a), row 1 shows ovarian cancer cell lines, row 2 shows primary ascites from ovarian cancer, row 3 shows colon, cervix and lung carcinoma, mesothelioma, row 4 shows HEK293 cells, row 5 shows negative cell lines derived from a T cell leukemia, a head and neck cancer and primary peripheral blood mononuclear cells (PBMCs) from a healthy donor).

The PRDX4-specific antibody 11F2 was shown to precipitate PRDX4 from lysates of cell lines that express surface PRDX4 or not (see FIG. 2b)). Cyanogen bromide beads were coupled with 11F2 and then incubated with 0.5 mg lysate in RIPA buffer at 4° C. overnight and were then centrifuged at 3.000 rpm for 5 min. Precipitated beads were incubated with Laemmli buffer, boiled for 5 min, and the eluates were separated by SDS-PAGE (lanes ‘IP’). Cell lysats were used as controls (L). Gels were blotted and the blots were incubated with a commercial PRDX4 antibody (Thermo Scientific), then with an HRP-labelled goat-anti rat antibody and were developed with ECL.

PRDX4 was biotinylated and thus present on the surface of cancer cells (see FIG. 2c)). Vital ES-2 tumor cells were incubated with membrane-impermeable EZ-Link Sulfo-NHS-SS-Biotin (Thermo Scientific) to exclusively biotinylate surface proteins. Then, cells were lysed and the lysate was incubated with 11F2-coupled beads as described above. Beads coupled with other antibodies were used as a control. Eluates of the precipitated beads were separated by SDS-PAGE. Immunoblots were performed with a streptavidin-HRP conjugate to detect biotinylated proteins (upper part) and with a commercial PRDX4 antibody (lower part). Blots were developed with ECL. Lysates from non-biotinylated cells (−) were used as controls. The first 2 lanes are cell lysates before IP.

### Example 3

### Binding of 11F2 to Vital ES-2 Cells

ES-2 cells were grown on microscope slides, washed in PBS and incubated with the PRDX4 antibody 11F2. Then, cells were fixed with 4% paraformaldehyde and incubated with an Alexa488-coupled suitable secondary antibody. The nuclei were counterstained with DAPI.

### Example 4

### Antibody 11F2 Precipitates PRDX4 and Binds within Exon 1 of PRDX4

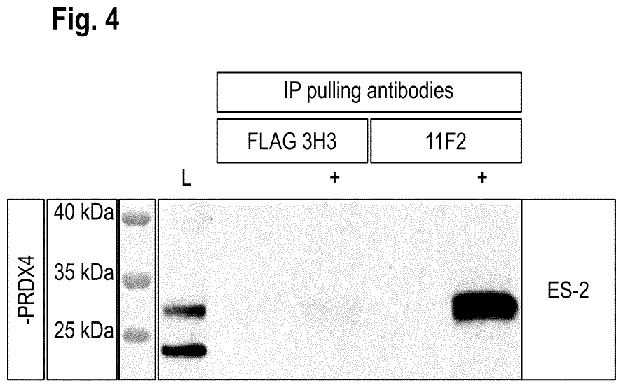

This example shows that the antibody 11F2 precipitates PRDX4 (see FIG. 4a)). Cyanogen bromide beads coupled with 11F2 were incubated overnight with lysates from ES-2 cells. After precipitation of the beads and incubation in Laemmli buffer, eluates were separated by SDS-PAGE and blotted. An immunoblot with a commercial PRDX4 antibody (Thermo Scientific) and a suitable secondary antibody revealed specific precipitation of PRDX4 by 11F2. Beads coupled with a FLAG-specific antibody were used as controls. L=cell lysate before immunoprecipitation.

This example further shows that 11F2 binds within exon 1 of PRDX4 (see FIG. 4b)). HEK293-cells were transfected with an expression plasmid encoding exon 1 (AS 1-80) of PRDX4 fused to GFP. 11F2 coupled to cyanogen bromide beads were incubated with lysates from transfectants or parental HEK293 cells. After precipitation, beads were incubated in Laemmli buffer and supernatants were separated by SDS-PAGE. An immunoblot with a GFP-specific antibody (Core Facility of the Helmholtz Centre Munich) and a suitable secondary antibody revealed the specificity of 11F2.

### Example 5

### PRDX4 is Membrane-Associated

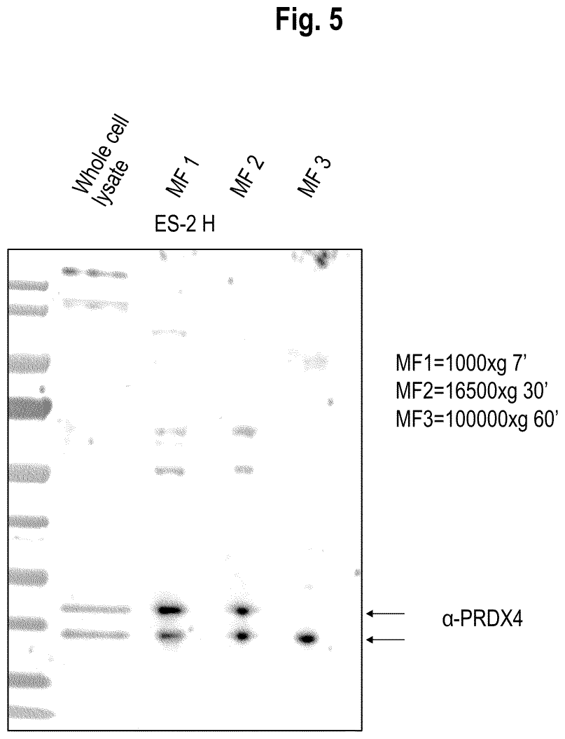

This example shows that PRDX4 is membrane-associated (see also FIG. 5). ES-2 cells were lysed and membrane fractions MF1 and MF2 were obtained by centrifugation at 7,000×g and 16,500×g, respectively. The cytosolic fraction, MF3, was obtained by centrifugation at 100,000×g.

### Example 6

### Surface-PRDX4-Positive SKOV3-Cells Activate PRDX4-Specific CAR-T-cells

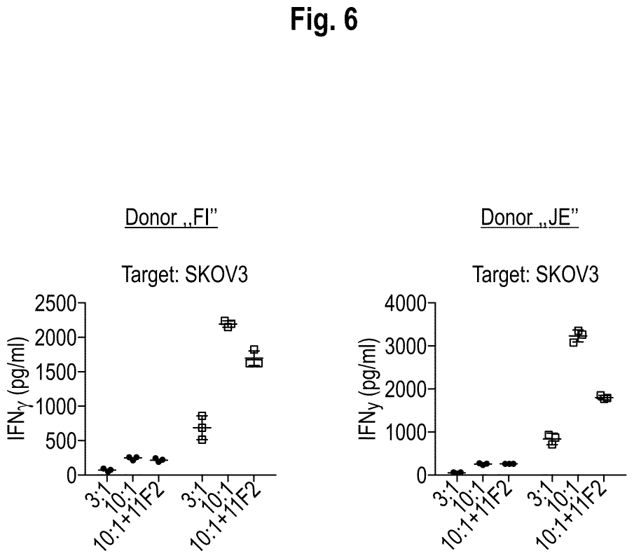

This example shows that surface-PRDX4-positive SKOV3-cells activate PRDX4-specific CAR-T-cells (see also FIG. 6). Primary T-cells from two donors (FI and JE) were transduced with a retroviral vector encoding a CAR construct with a single-chain construct derived from antibody 11F2. Expression of the CAR was tested by flow cytometry with a goat-anti rat IgG antibody (not shown). SKOV3 cells were incubated with transduced T cells (columns 4-6 of the blots) of non-transduced control T cells (column 1-3 of the blots) at the ratios indicated on the X-axis for 24 h. Then, interferon-gamma in the supernatants, as a marker for T-cell activation, was quantified with a commercial ELISA assay. As an additional proof of specificity, some of the co-cultures (columns 3 and 6) were pre-incubated with 11F2 antibody in order to block recognition of target cells by the T cells. This pre-incubation resulted in a reduced interferon concentration in the supernatants.

### Example 7

### 11F2 is PRDX4-Specific

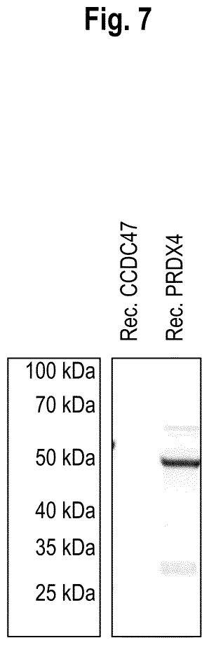

This example shows that 11F2 is PRDX4-specific (see also FIG. 7). 11F2 coupled to cyanogen bromide beads were incubated with 10 ng of recombinant PRDX4 or, as a control, with the same amount of recombinant CCDC47 protein. After precipitation, beads were incubated in Laemmli buffer and supernatants were separated by SDS-PAGE. An immunoblot with a commercial PRDX4 antibody (Thermo Scientific) and a suitable secondary antibody revealed the specificity of 11F2. The recombinant PRDX4 protein is a dimer and thus about 50 kD in size.

### Example 8

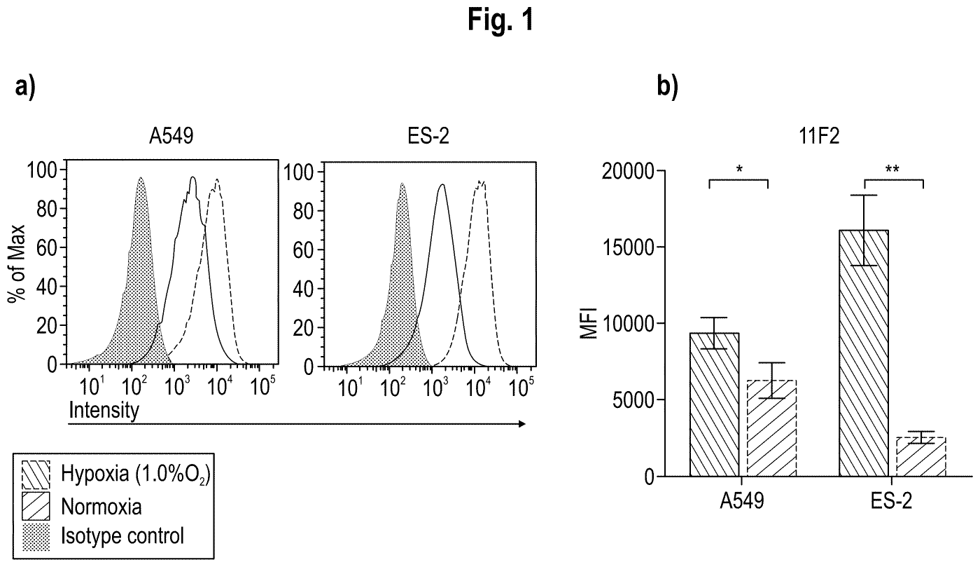

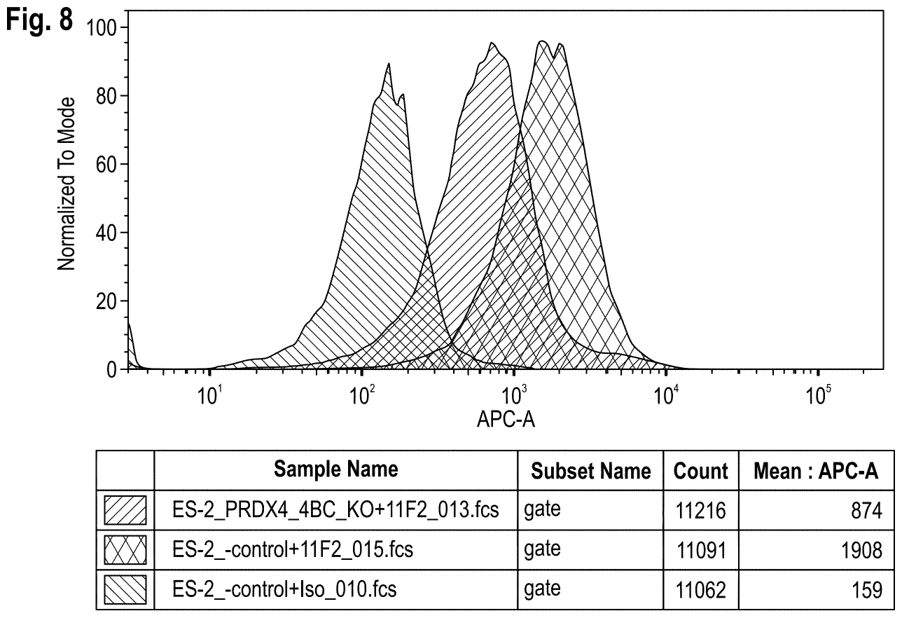

It was also proven by FIG. 8 that 11F2 is PRDX4-specific: ES-2 cells were transfected with PRDX4-specific siRNAs or a control siRNA. 3 days later, binding of 11F2 was measured by flow cytometry as described in FIG. 1. Shown is that the siRNA-transfected population 4BC_KO only reveals approx. 50% of 11F2 binding (mean =874) as compared to control-siRNA transfected ES-2 w cells (mean =1908). An isotype antibody was included as a control (mean=159).

### Example 9

PRDX4-Specific Antibodies Present in Ascites from Patients with Ovarian Cancer

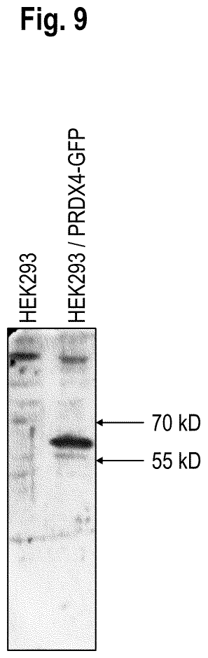

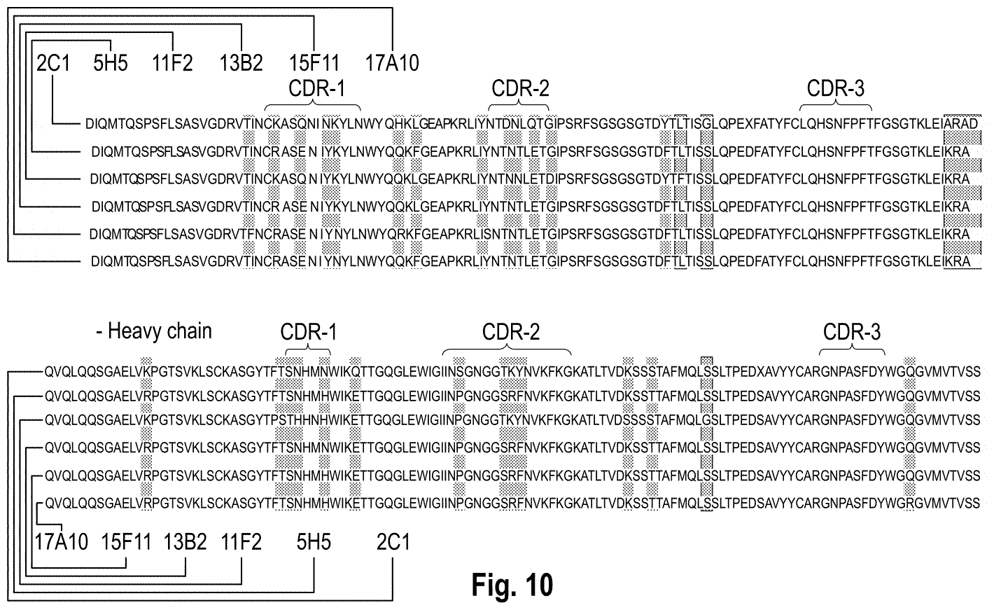

PRDX4-specific antibodies were present in ascites from patients with ovarian cancer (see FIG. 9). HEK293-cells were transfected with an expression plasmid encoding a PRDX4-GFP fusion protein, approx. 55 kD in size. Similar amounts of lysates of transfectants and parental HEK293 cells were separated by SDS-PAGE and blotted. This blot was then incubated with ascites, followed by incubation with a goat-anti human IgG antibody and developed with ECL.

### REFERENCES

Fujii, J., Ikeda, Y., Kurahashi, T., and Homma, T. (2015). Physiological and pathological views of peroxiredoxin 4. Free Radic Biol Med 83, 373-379.

Hashimoto-Gotoh, T. et al. (1995) Gene 152, 271-275.

Hwang, J. A., Song, J. S., Yu, D. Y., Kim, H. R., Park, H. J., Park, Y. S., Kim, W. S., and Choi, C. M. (2015). Peroxiredoxin 4 as an independent prognostic marker for survival in patients with early-stage lung squamous cell carcinoma. Int J Clin Exp Pathol 8, 6627-6635.

Jeong, S. H., Kim, R. B., Park, S. Y., Park, J., Jung, E. J., Ju, Y. T., Jeong, C. Y., Park, M., Ko, G. H., Song, D. H., Koh, H. M., Kim, W. H., Yang, H. K., Lee, Y. J., and Hong, S. C. (2020). Nomogram for predicting gastric cancer recurrence using biomarker gene expression. Eur J Surg Oncol 46, 195-201.

Kramer, W. et al. (1984) Nucleic Acids Res. 12, 9441-9456.

Kunkel, T A (1985) Proc Natl Acad Sci USA. 82, 488-492.

Kunkel (1988) Methods Enzymol. 85, 2763-2766.

Mark, D. F. et al., Proc. Natl. Acad. Sci. USA (1984) 81, 5662-5666, Zoller, M. J. and Smith, M., Nucleic Acids Research (1982) 10, 6487-6500.

Mizutani, K., Guo, X., Shioya, A., Zhang, J., Zheng, J., Kurose, N., Ishibashi, H., Motono, N., Uramoto, H., and Yamada, S. (2019). The impact of PRDX4 and the EGFR mutation status on cellular proliferation in lung adenocarcinoma. Int J Med Sci 16, 1199-1206.

Schulte, J. (2011). Peroxiredoxin 4: a multifunctional biomarker worthy of further exploration. BMC Med 9, 137.

Schulte, J., Struck, J., Köhrle, J., and Müller, B. (2011). Circulating levels of peroxiredoxin 4 as a novel biomarker of oxidative stress in patients with sepsis. Shock 35, 460-465.

Wang, G., Zhong, W. C., Bi, Y. H., Tao, S. Y., Zhu, H., Zhu, H. X., and Xu, A. M. (2019). The Prognosis Of Peroxiredoxin Family In Breast Cancer. Cancer Manag Res 11, 9685-9699.

Wang, A. et al., Science 224, 1431-1433, Dalbadie-McFarland, G. et al., Proc. Natl. Acad. Sci. USA (1982) 79, 6409-641.

Weidle, U. H., Maisel, D., Klostermann, S., Schiller, C., and Weiss, E. H. (2011). Intracellular proteins displayed on the surface of tumor cells as targets for therapeutic intervention with antibody-related agents. Cancer Genomics Proteomics 8, 49-63.

Zoller, M J, and Smith, M. (1983) Methods Enzymol. 100, 468-500.

EP-Al 2546269

1. Pharmaceutical composition comprising a monoclonal antibody which binds to human PRDX4 present on the cell surface of a cancer cell and optionally a pharmaceutically acceptable carrier, diluent or excipient, wherein said antibody recognizes a region from amino acids 81 to 271 in human PRDX4 shown in SEQ ID NO: 81.

2. The pharmaceutical composition of claim 1, wherein said antibody has cytotoxic activity.

3. The pharmaceutical composition of any one of the preceding claims, wherein said antibody has ADCC or CDC.

4. The pharmaceutical composition of any one of the preceding claims, wherein said antibody is conjugated with a cytotoxic substance.

5. The pharmaceutical composition of any one of the preceding claims, wherein said antibody is part of a chimeric antigen receptor (CAR).

6. The pharmaceutical composition of claim 5, wherein said CAR is comprised by a T cell, NK cell, NK-T cell or macrophage.

7. The pharmaceutical composition of any one of the preceding claims, wherein said antibody is less than 20% cross-reactive with PRDX4-related proteins.

8. The pharmaceutical composition of any one of the preceding claims, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16;
(i) an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(j) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

9. The pharmaceutical composition of any one of the preceding claims, wherein said antibody is
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23, and
a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62,
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68,
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74,
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80, or
(k) an antibody which binds to the same epitope as that in the human PRDX4 protein to which any one of the antibodies (a) to (j) bind.

10. The pharmaceutical composition of any one of the preceding claims for use in a method for the treatment of cancer in a human subject.

11. The pharmaceutical composition for the use of claim 10, wherein said cancer is characterized by cells, wherein human PRDX4 is present on their cell surface.

12. The pharmaceutical composition for the use of claim 10 or 11, wherein said cancer is breast cancer, prostate cancer, lung cancer, colorectal cancer, ovarian cancer, oral cavity squamous carcinoma, hematologic malignancy tumor, or glioma.

13. A method for determining whether a human subject may suffer from cancer, comprising determining in a sample obtained from said human subject whether PRDX4 is present on the cell surface of cancer cells comprised by said sample.

14. An antibody which binds to human PRDX4 present on the cell surface of a cancer cell, wherein said antibody is
(a) an antibody comprising a heavy chain variable region having an amino acid sequence with at least 85% identity to the amino acid sequence shown in SEQ ID NO: 1 and a light chain variable region having an amino acid sequence with at least 85% identity to the amino acid sequence shown in SEQ ID NO: 2;
(b) an antibody comprising a heavy chain variable region having an amino acid sequence with at least 85% identity to the amino acid sequence shown in SEQ ID NO: 3 and a light chain variable region having an amino acid sequence with at least 85% identity to the amino acid sequence shown in SEQ ID NO: 4;
(c) an antibody comprising a heavy chain variable region having an amino acid sequence with at least 85% identity to the amino acid sequence shown in SEQ ID NO: 5 and a light chain variable region having an amino acid sequence with at least 85% identity to the amino acid sequence shown in SEQ ID NO: 6;
(d) an antibody comprising a heavy chain variable region having an amino acid sequence with at least 85% identity to the amino acid sequence shown in SEQ ID NO: 7 and a light chain variable region having an amino acid sequence with at least 85% identity to the amino acid sequence shown in SEQ ID NO: 8;
(e) an antibody comprising a heavy chain variable region having an amino acid sequence with at least 85% identity to the amino acid sequence shown in SEQ ID NO: 9 and a light chain variable region having an amino acid sequence with at least 85% identity to the amino acid sequence shown in SEQ ID NO: 10;
(f) an antibody comprising a heavy chain variable region having an amino acid sequence with at least 85% identity to the amino acid sequence shown in SEQ ID NO: 11 and a light chain variable region having an amino acid sequence with at least 85% identity to the amino acid sequence shown in SEQ ID NO: 12;
(g) an antibody comprising a heavy chain variable region having an amino acid sequence with at least 85% identity to the amino acid sequence shown in SEQ ID NO: 13 and a light chain variable region having an amino acid sequence with at least 85% identity to the amino acid sequence shown in SEQ ID NO: 14;
(h) an antibody comprising a heavy chain variable region having an amino acid sequence with at least 85% identity to the amino acid sequence shown in SEQ ID NO: 15 and a light chain variable region having an amino acid sequence with at least 85% identity to the amino acid sequence shown in SEQ ID NO: 16;
(i) an antibody comprising a heavy chain variable region having an amino acid sequence with at least 85% identity to the amino acid sequence shown in SEQ ID NO: 17 and a light chain variable region having an amino acid sequence with at least 85% identity to the amino acid sequence shown in SEQ ID NO: 18;
(j) an antibody comprising a heavy chain variable region having an amino acid sequence with at least 85% identity to the amino acid sequence shown in SEQ ID NO: 19 and a light chain variable region having an amino acid sequence with at least 85% identity to the amino acid sequence shown in SEQ ID NO: 20; or
(k) an antibody which binds to the same epitope as that in the human PRDX4 protein to which any one of the antibodies (a) to (j) bind.

15. An antibody which binds to human PRDX4 present on the cell surface of a cancer cell comprising
(a) an antibody comprising a heavy chain variable region comprising heavy chain CDR1 having the amino acid sequence as set forth in SEQ ID NO: 21, heavy chain CDR2 having the amino acid sequence as set forth in SEQ ID NO: 22, and heavy chain CDR3 having the amino acid sequence as set forth in SEQ ID NO: 23 and
a light chain variable region comprising light chain CDR1 having the amino acid sequence as set forth in SEQ ID NO: 24, light chain CDR2 having the amino acid sequence as set forth in SEQ ID NO: 25, and light chain CDR3 having the amino acid sequence as set forth in SEQ ID NO: 26;
(b) an antibody comprising a heavy chain variable region comprising heavy chain CDR1 having the amino acid sequence as set forth in SEQ ID NO: 27, heavy chain CDR2 having the amino acid sequence as set forth in SEQ ID NO: 28, and heavy chain CDR3 having the amino acid sequence as set forth in SEQ ID NO: 29, and
a light chain variable region comprising light chain CDR1 having the amino acid sequence as set forth in SEQ ID NO: 30, light chain CDR2 having the amino acid sequence as set forth in SEQ ID NO: 31, and light chain CDR3 having the amino acid sequence as set forth in SEQ ID NO: 32;
(c) an antibody comprising a heavy chain variable region comprising heavy chain CDR1 having the amino acid sequence as set forth in SEQ ID NO: 33, heavy chain CDR2 having the amino acid sequence as set forth in SEQ ID NO: 34, and heavy chain CDR3 having the amino acid sequence as set forth in SEQ ID NO: 35, and
a light chain variable region comprising light chain CDR1 having the amino acid sequence as set forth in SEQ ID NO: 36, light chain CDR2 having the amino acid sequence as set forth in SEQ ID NO: 37, and light chain CDR3 having the amino acid sequence as set forth in SEQ ID NO: 38;
(d) an antibody comprising a heavy chain variable region comprising heavy chain CDR1 having the amino acid sequence as set forth in SEQ ID NO: 39, heavy chain CDR2 having the amino acid sequence as set forth in SEQ ID NO: 40, and heavy chain CDR3 having the amino acid sequence as set forth in SEQ ID NO: 41, and
a light chain variable region comprising light chain CDR1 having the amino acid sequence as set forth in SEQ ID NO: 42, light chain CDR2 having the amino acid sequence as set forth in SEQ ID NO: 43, and light chain CDR3 having the amino acid sequence as set forth in SEQ ID NO: 44;
(e) an antibody comprising a heavy chain variable region comprising heavy chain CDR1 having the amino acid sequence as set forth in SEQ ID NO: 45, heavy chain CDR2 having the amino acid sequence as set forth in SEQ ID NO: 46, and heavy chain CDR3 having the amino acid sequence as set forth in SEQ ID NO: 47, and
a light chain variable region comprising light chain CDR1 having the amino acid sequence as set forth in SEQ ID NO: 48, light chain CDR2 having the amino acid sequence as set forth in SEQ ID NO: 49, and light chain CDR3 having the amino acid sequence as set forth in SEQ ID NO: 50;
(f) an antibody comprising a heavy chain variable region comprising heavy chain CDR1 having the amino acid sequence as set forth in SEQ ID NO: 51, heavy chain CDR2 having the amino acid sequence as set forth in SEQ ID NO: 52, and heavy chain CDR3 having the amino acid sequence as set forth in SEQ ID NO: 53, and
a light chain variable region comprising light chain CDR1 having the amino acid sequence as set forth in SEQ ID NO: 54, light chain CDR2 having the amino acid sequence as set forth in SEQ ID NO: 55, and light chain CDR3 having the amino acid sequence as set forth in SEQ ID NO: 56;
(g) an antibody comprising a heavy chain variable region comprising heavy chain CDR1 having the amino acid sequence as set forth in SEQ ID NO: 57, heavy chain CDR2 having the amino acid sequence as set forth in SEQ ID NO: 58, and heavy chain CDR3 having the amino acid sequence as set forth in SEQ ID NO: 59, and
a light chain variable region comprising light chain CDR1 having the amino acid sequence as set forth in SEQ ID NO: 60, light chain CDR2 having the amino acid sequence as set forth in SEQ ID NO: 61, and light chain CDR3 having the amino acid sequence as set forth in SEQ ID NO: 62;
(h) an antibody comprising a heavy chain variable region comprising heavy chain CDR1 having the amino acid sequence as set forth in SEQ ID NO: 63, heavy chain CDR2 having the amino acid sequence as set forth in SEQ ID NO: 64, and heavy chain CDR3 having the amino acid sequence as set forth in SEQ ID NO: 65, and
a light chain variable region comprising light chain CDR1 having the amino acid sequence as set forth in SEQ ID NO: 66, light chain CDR2 having the amino acid sequence as set forth in SEQ ID NO: 67, and light chain CDR3 having the amino acid sequence as set forth in SEQ ID NO: 68;
(i) an antibody comprising a heavy chain variable region comprising heavy chain CDR1 having the amino acid sequence as set forth in SEQ ID NO: 69, heavy chain CDR2 having the amino acid sequence as set forth in SEQ ID NO: 70, and heavy chain CDR3 having the amino acid sequence as set forth in SEQ ID NO: 71, and
a light chain variable region comprising light chain CDR1 having the amino acid sequence as set forth in SEQ ID NO: 72, light chain CDR2 having the amino acid sequence as set forth in SEQ ID NO: 73, and light chain CDR3 having the amino acid sequence as set forth in SEQ ID NO: 74;
(j) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77, and
a light chain variable region comprising light chain CDR1 having the amino acid sequence as set forth in SEQ ID NO: 78, light chain CDR2 having the amino acid sequence as set forth in SEQ ID NO: 79, and light chain CDR3 having the amino acid sequence as set forth in SEQ ID NO: 80; or
(k) an antibody which binds to the same epitope as that in the human PRDX4 protein to which any one of the antibodies (a) to (j) bind.


SEQUENCE LISTING (82 sequences)

SEQ ID NO: 1 - protein, 117 aa, artificial
  2C1 VH region
QVQLQQSGAELVKPGTSVKLSCKASGYTFTSNHMNWIKQTTGQGLEWIGIINSGNGGTKYNVKFKGKATLTVDKSSSTAFMQLSSLTPEDSAVYYCARGNPASFDYWGQGVMVTVSS

SEQ ID NO: 2 - protein, 110 aa, artificial
  2C1 VL region
DIQMTQSPSFLSASVGDRVTINCKASQNINKYLNWYQHKLGEAPKRLIYNTDNLQTGIPSRFSGSGSGTDYTLTISGLQPEDFATYFCLQHSNFPFTFGSGTKLEIARAD

SEQ ID NO: 3 - protein, 117 aa, artificial
  5H5 VH region
QVQLQQSGAELVRPGTSVKLSCKASGYTFTSNHMHWIKETTGQGLEWIGIINPGNGGSRFNVKFKGKATLTVDKSSTTAFMQLSSLTPEDSAVYYCARGNPASFDYWGQGVMVTVSS

SEQ ID NO: 4 - protein, 109 aa, artificial
  5H5 VL region
DIQMTQSPSFLSASVGDRVTINCRASENIYKYLNWYQQKFGEAPKRLIYNTNTLETGIPSRFSGSGSGTDFTLTISSLQPEDFATYFCLQHSNFPFTFGSGTKLEIKRA

SEQ ID NO: 5 - protein, 117 aa, artificial
  10G7 VH region
QVQLQQSGAELVKPGTSVKLSCKASGYTFTSNHMNWIKQTTGQGLEWIGIINPVNGGTRYNVKFKGKATLTVDKSSSTAFMQLSSLTPEDSAVYYCARGNPASFDYWGQGVMVTVSS

SEQ ID NO: 6 - protein, 109 aa, artificial
  10G7 VL region
DIQMTQSPSFLSASVGDRVTINCKASQNIHKYLNWYQHKLGEAPKRLIYNTNNLQTDIPSNFSGSGSGTDYTLTISSLQPEDFATYFCLQHSSFPFTFGSGTKLEIKRA

SEQ ID NO: 7 - protein, 117 aa, artificial
  11-F2 VH region
QVQLQQSGAELVKPGTSVKLSCKASGYTFSTHHMHWIKETTGQGLEWIGIINPGNGGTKYNVKFKGKATLTVDSSSSTAFMQLGSLTPEDSAVYYCARGNPASFDYWGQGVMVTVSS

SEQ ID NO: 8 - protein, 109 aa, artificial
  11-F2 VL region
DIQMTQSPSFLSASVGDRVTINCKASQNIYKYLNWYQQKLGEAPKRLIYNTNNLETDIPSRFSGSGSGTDYTFTISSLQPEDFATYFCLQHSNFPFTFGSGTKLEIKRA

SEQ ID NO: 9 - protein, 117 aa, artificial
  13B2 VH region
QVQLQQSGAELVRPGTSVKLSCKASGYTFTSNHMNWIKETTGQGLEWIGIINPGNGGSRFNVKFKGKATLTVDKSSTTAFMQLSSLTPEDSAVYYCARGNPASFDYWGQGVMVTVSS

SEQ ID NO: 10 - protein, 109 aa, artificial
  13B2 VL region
DIQMTQSPSFLSASVGDRVTINCRASENIYKYLNWYQQKLGEAPKRLIYNTNTLETGIPSRFSGSGSGTDFTLTISSLQPEDFATYFCLQHSNFPFTFGSGTKLEIKRA

SEQ ID NO: 11 - protein, 117 aa, artificial; identical to SEQ ID NO: 3
  15-F11 VH region

SEQ ID NO: 12 - protein, 109 aa, artificial
  15-F11 VL region
DIQMTQSPSFLSASVGDRVTFNCRASENIYNYLNWYQRKFGEAPKRLISNTNTLETGIPSRFSGSGSGTDFTLTISSLQPEDFATYFCLQHSNFPFTFGSGTKLEIKRA

SEQ ID NO: 13 - protein, 117 aa, artificial
  17A10 VH region
QVQLQQSGAELVRPGTSVKLSCKASGYTFTSNHMHWIKETTGQGLEWIGIINPGNGGSRFNVKFKGKATLTVDKSYTTAFMQLSSLTPEDSAVYYCARGNPASFDYWGRGVMVTVSS

SEQ ID NO: 14 - protein, 109 aa, artificial
  17A10 VL region
DIQMTQSPSFLSASVGDRVTINCRASENIYNYLNWYQQKFGEAPKRLIYNTNTLETGIPSRFSGSGSGTDFTLTISSLQPEDFATYFCLQHSNFPFTFGSGTKLEIKRA

SEQ ID NO: 15 - protein, 117 aa, artificial
  18G8 VH region
QVQLQQSGAELVEPGTSVRLSCKASGYTFTNNHIHWIKQATGQALEWIGIINSGNGGTKYDVKFKGKATLTVDKSSNTAFMQLSSLTPEDSAVYYCARGNPASFDYWGQGVLVTVSS

SEQ ID NO: 16 - protein, 109 aa, artificial
  18G8 VL region
DIQMTQSPSFLSASVGDRLTINCKASQNIYKYLNWYQQKLGEAPKRLIYDTNNLQTGIPSRFSGSGSGTDYTLTISSLQPEDFATYFCLQHSNFPFTFGSGTKLEIKRA

SEQ ID NO: 17 - protein, 117 aa, artificial
  19H9 VH region
QVQLQQSGVELVRPGTSVRLSCKASGYTFTSNHMHWIKETTGQGLEWIGIINPGNGGSRFNVKFKGKATLTVDKSSTTAFMQLNSLTPEDSAVYYCARGNPASFDYWGQGVMVTVSS

SEQ ID NO: 18 - protein, 109 aa, artificial
  19H9 VL region
DIQMTQSPSFLSASVGDRVTINCRASENIYNYLNWYQQKFGEGPKRLIYNTNTLETGIPSRFSGSGSGTDFTLTINSLQPEDFATYFCLQHSNFPFTFGSGTKLEIKRA

SEQ ID NO: 19 - protein, 117 aa, artificial
  24B7 VH region
QVQLQQSGAELVRPGTSVKLSCKASGYTFTSNHMHWIKETTGQGLEWIGIINPGNGGSRFNVKFKGKATLTVDKSSTTAFMQLSSLTPEDSAVYYCARGNPASFDYWGRGVMVTVSS

SEQ ID NO: 20 - protein, 109 aa, artificial; identical to SEQ ID NO: 14
  24B7 VH region

SEQ ID NO: 21 - protein, 6 aa, artificial
  2C1 VH-CDR1
TSNHMN

SEQ ID NO: 22 - protein, 17 aa, artificial
  2C1 VH-CDR2
IINSGNGGTKYNVKFKG

SEQ ID NO: 23 - protein, 8 aa, artificial
  2C1 VH-CDR3
GNPASFDY

SEQ ID NO: 24 - protein, 11 aa, artificial
  2C1 VL-CDR1
KASQNINKYLN

SEQ ID NO: 25 - protein, 7 aa, artificial
  2C1 VL-CDR2
NTDNLQT

SEQ ID NO: 26 - protein, 9 aa, artificial
  2C1 VL-CDR3
LQHSNFPFT

SEQ ID NO: 27 - protein, 6 aa, artificial
  5H5 VH-CDR1
TSNHMH

SEQ ID NO: 28 - protein, 17 aa, artificial
  5H5 VH-CDR2
IINPGNGGSRFNVKFKG

SEQ ID NO: 29 - protein, 8 aa, artificial
  5H5 VH-CDR3
GNPASFDY

SEQ ID NO: 30 - protein, 11 aa, artificial
  5H5 VL-CDR1
RASENIYKYLN

SEQ ID NO: 31 - protein, 8 aa, artificial
  5H5 VL-CDR2
NTNTLETG

SEQ ID NO: 32 - protein, 9 aa, artificial
  5H5 VL-CDR3
LQHSNFPFT

SEQ ID NO: 33 - protein, 6 aa, artificial
  10G7 VH-CDR1
TSNHMN

SEQ ID NO: 34 - protein, 17 aa, artificial
  10G7 VH-CDR2
IINPVNGGTRYNVKFKG

SEQ ID NO: 35 - protein, 8 aa, artificial
  10G7 VH-CDR3
GNPASFDY

SEQ ID NO: 36 - protein, 11 aa, artificial
  10G7 VL-CDR1
KASQNIHKYLN

SEQ ID NO: 37 - protein, 7 aa, artificial
  10G7 VL-CDR2
NTNNLQT

SEQ ID NO: 38 - protein, 9 aa, artificial
  10G7 VL-CDR3
LQHSSFPFT

SEQ ID NO: 39 - protein, 6 aa, artificial
  11F2 VH-CDR1
STHHMH

SEQ ID NO: 40 - protein, 17 aa, artificial
  11F2 VH-CDR2
IINPGNGGTKYNVKFKG

SEQ ID NO: 41 - protein, 8 aa, artificial
  11F2 VH-CDR3
GNPASFDY

SEQ ID NO: 42 - protein, 11 aa, artificial
  11F2 VL-CDR1
KASQNIYKYLN

SEQ ID NO: 43 - protein, 9 aa, artificial
  11F2 VL-CDR2
YNTNNLETD

SEQ ID NO: 44 - protein, 9 aa, artificial
  11F2 VL-CDR3
LQHSNFPFT

SEQ ID NO: 45 - protein, 6 aa, artificial
  13B2 VH-CDR1
TSNHMN

SEQ ID NO: 46 - protein, 17 aa, artificial
  13B2 VH-CDR2
IINPGNGGSRFNVKFKG

SEQ ID NO: 47 - protein, 8 aa, artificial
  13B2 VH-CDR3
GNPASFDY

SEQ ID NO: 48 - protein, 11 aa, artificial
  13B2 VL-CDR1
RASENIYKYLN

SEQ ID NO: 49 - protein, 9 aa, artificial
  13B2 VL-CDR2
YNTNTLETG

SEQ ID NO: 50 - protein, 9 aa, artificial
  13B2 VL-CDR3
LQHSNFPFT

SEQ ID NO: 51 - protein, 6 aa, artificial
  15F11 VH-CDR1
TSNHMH

SEQ ID NO: 52 - protein, 17 aa, artificial
  15F11 VH-CDR2
IINPGNGGSRFNVKFKG

SEQ ID NO: 53 - protein, 8 aa, artificial
  15F11 VH-CDR3
GNPASFDY

SEQ ID NO: 54 - protein, 11 aa, artificial
  15F11 VL-CDR1
RASENIYNYLN

SEQ ID NO: 55 - protein, 9 aa, artificial
  15F11 VL-CDR2
SNTNTLETG

SEQ ID NO: 56 - protein, 9 aa, artificial
  15F11 VL-CDR3
LQHSNFPFT

SEQ ID NO: 57 - protein, 6 aa, artificial
  17A10 VH-CDR1
TSNHMH

SEQ ID NO: 58 - protein, 17 aa, artificial
  17A10 VH-CDR2
IINPGNGGSRFNVKFKG

SEQ ID NO: 59 - protein, 8 aa, artificial
  17A10 VH-CDR3
GNPASFDY

SEQ ID NO: 60 - protein, 11 aa, artificial
  17A10 VL-CDR1
RASENIYNYLN

SEQ ID NO: 61 - protein, 9 aa, artificial
  17A10 VL-CDR2
YNTNTLETG

SEQ ID NO: 62 - protein, 9 aa, artificial
  17A10 VL-CDR3
LQHSNFPFT

SEQ ID NO: 63 - protein, 6 aa, artificial
  18G8 VH-CDR1
TNNHIH

SEQ ID NO: 64 - protein, 17 aa, artificial
  18G8 VH-CDR2
IINSGNGGTKYDVKFKG

SEQ ID NO: 65 - protein, 8 aa, artificial
  18G8 VH-CDR3
GNPASFDY

SEQ ID NO: 66 - protein, 11 aa, artificial
  18G8 VL-CDR1
KASQNIYKYLN

SEQ ID NO: 67 - protein, 8 aa, artificial
  18G8 VL-CDR2
DTNNLQTG

SEQ ID NO: 68 - protein, 9 aa, artificial
  18G8 VL-CDR3
LQHSNFPFT

SEQ ID NO: 69 - protein, 6 aa, artificial
  19H9 VH-CDR1
TSNHMH

SEQ ID NO: 70 - protein, 17 aa, artificial
  19H9 VH-CDR2
IINPGNGGSRFNVKFKG

SEQ ID NO: 71 - protein, 8 aa, artificial
  19H9 VH-CDR3
GNPASFDY

SEQ ID NO: 72 - protein, 11 aa, artificial
  19H9 VL-CDR1
RASENIYNYLN

SEQ ID NO: 73 - protein, 8 aa, artificial
  19H9 VL-CDR2
NTNTLETG

SEQ ID NO: 74 - protein, 9 aa, artificial
  19H9 VL-CDR3
LQHSNFPFT

SEQ ID NO: 75 - protein, 6 aa, artificial
  24B7 VH-CDR1
TSNHMH

SEQ ID NO: 76 - protein, 17 aa, artificial
  24B7 VH-CDR2
IINPGNGGSRFNVKFKG

SEQ ID NO: 77 - protein, 8 aa, artificial
  24B7 VH-CDR3
GNPASFDY

SEQ ID NO: 78 - protein, 11 aa, artificial
  24B7 VL-CDR1
RASENIYNYLN

SEQ ID NO: 79 - protein, 8 aa, artificial
  24B7 VL-CDR2
NTNTLETG

SEQ ID NO: 80 - protein, 9 aa, artificial
  24B7 VL-CDR3
LQHSNFPFT

SEQ ID NO: 81 - protein, 271 aa, human
MEALPLLAATTPDHGRHRRLLLLPLLLFLLPAGAVQGWETEERPRTREEECHFYAGGQVYPGEASRVSVADHSLHLSKAKISKPAPYWEGTAVIDGEFKELKLTDYRGKYLVFFFYPLDFTFVCPTEIIAFGDRLEEFRSINTEVVACSVDSQFTHLAWINTPRRQGGLGPIRIPLLSDLTHQISKDYGVYLEDSGHTLRGLFIIDDKGILRQITLNDLPVGRSVDETLRLVQAFQYTDKHGEVCPAGWKPGSETIIPDPAGKLKYFDKLN

SEQ ID NO: 82 - protein, 9 aa, human
WETEERPRT